(19) United States
(12) Patent Application Publication
(10) Pub. No.: US 2025/0098996 A1
(43) Pub. Date: Mar. 27, 2025

(54) WEARABLE ELECTRONIC RING, RESPONSIVE SERVER SYSTEM, HEALTH MONITORING, NOTIFICATION AND RECOMMENDATION METHOD AND MEDIUM

(71) Applicant: Armin Hoes, Los Angeles, CA (US)

(72) Inventor: Armin Hoes, Los Angeles, CA (US)

(21) Appl. No.: 18/373,476

(22) Filed: Sep. 27, 2023

(51) Int. Cl. A61B 5/16; A44C 9/00; G06F 1/16

(52) U.S. Cl. CPC A61B 5/165; A44C 9/0053; G06F 1/163

(57) ABSTRACT

Systems and methods including controlling a wearable ring display to display a first color corresponding to a first mood or emotion at a first point in time, controlling the wearable ring display to display a second color corresponding to a second mood or emotion at a second point in time. The first color is different than the second color, and the displayed ring color is associated with a current feeling or mood.

### FIELD

The disclosure relates to embodiments relate generally to wearable devices, systems, and methods, and more particularly are directed to wearable interactive electronic display devices attachable to humans or animals.

### BACKGROUND

Various methods have been proposed for health and wellness monitoring via wearable electronic devices. Wearable devices are directed to one or more physiological sensors of the wearable device being in contact with the skin of the user.

Thus, there is a need for improvements to wearable devices that enhance accuracy of health and wellness monitoring. There are other shortcomings of the methods in the related art, as discussed below.

The above information is presented as background information only to assist with an understanding of the disclosure. No determination has been made, and no assertion is made, as to whether any of the above might be applicable as prior art regarding the disclosure.

### SUMMARY

Aspects of the disclosure are to address at least the above-mentioned and below-mentioned problems and/or disadvantages and to provide at least the advantages described below. As discussed above, there is a need for improvements to wearable devices for health and wellness monitoring.

According to various embodiments, the present disclosure provides features and components to: (i) increase user friendliness/ease of use of wearable ring devices, (ii) provide real time notifications/alerts via wearable ring devices, and (iii) enhance the accuracy of health and wellness monitoring performed, at least in part, by wearable devices.

Various embodiments also include smart technology applications for control and operations of the system by electronic devices (e.g., a smartphone).

Accordingly, an aspect of the disclosure is to provide a method including: detecting, by an electronic ring device, a user interaction with a button of the electronic ring device; and based on the detecting of the user interaction with the button, performing a ring control operation that controls the electronic ring device. According to an embodiment, the performing of the ring control operation includes automatically controlling the ring to change a display from a first color to a second color that is different from the first color.

An aspect of the disclosure is to provide a wearable electronic ring device comprising: a wearable ring body housing, wherein the body housing has a cylindrical shape and a first outer surface configured to engage with a digit of a dactyl mammal as a ring, and the first outer surface is firmly attachable to the digit of the dactyl mammal as the ring; a wearable ring display; a wearable ring memory; and wearable ring processing circuitry, wherein the wearable ring processing circuitry is configured to execute computer-executable instructions stored in the wearable ring memory to perform the following operations: controlling the wearable ring display to display a first color corresponding to a first mood or emotion at a first point in time; and controlling the wearable ring display to display a second color corresponding to a second mood or emotion at a second point in time, wherein the first color is different than the second color, wherein the displayed ring color is associated with a current feeling or mood.

The operations may further include a wearable ring network communication interface and a wearable ring button provided on a second outer surface of the wearable ring housing, wherein the computer-executable instructions stored in the wearable ring memory further include instructions configured to cause the wearable ring processing circuitry to perform the following operations: responsive to receiving a user ring input via the wearable ring button, controlling the network communication interface to transmit the user ring input to a responsive server; receive, via the network communication interface, a response to the transmitted user ring input from the server; and responsive to receiving the response to the transmitted user ring input from the server, control the wearable ring display to display the first color corresponding to the first mood or the second color corresponding to the second mood based on the received response to the transmitted user ring input.

The controlling of the wearable ring display to display the first color corresponding to the first mood or the second color corresponding to the second mood based on the received response to the transmitted user ring input may include controlling the wearable ring display to change a color of the wearable ring display. The controlling the wearable ring display to change a color of the wearable ring display may include outputting a notification indicating the color change. The outputting of the notification indicating the color change includes at least one of: controlling the wearable ring display to flash, controlling the wearable ring display to pulse, or controlling the wearable ring to output haptic feedback.

An aspect of the disclosure is to provide a real time responsive server method comprising: controlling a wearable ring display to display a first color corresponding to a first mood or emotion at a first point in time; and controlling the wearable ring display to display a second color corresponding to a second mood or emotion at a second point in time, wherein the first color is different than the second color, wherein the displayed ring color is associated with a current feeling.

The operations of the real time server method may further comprise: responsive to receiving a user ring input via a wearable ring button, controlling a network communication interface of a wearable ring device to transmit the user ring input to a responsive server; receiving, via the network communication interface, a response to the transmitted user ring input from the server; and responsive to receiving the response to the transmitted user ring input from the server, controlling the wearable ring display to display the first color corresponding to the first mood or the second color corresponding to the second mood based on the received response to the transmitted user ring input.

The controlling of the wearable ring display to display the first color corresponding to the first mood or the second color corresponding to the second mood based on the received response to the transmitted user ring input may include controlling the wearable ring display to change a color of the wearable ring display.

The real time responsive server method may further comprise: receiving, from the wearable ring device, a packet including raw digit activity (RDA); identifying, based on the packet, a registered wearable device user (RWDU) associated with a wearable ring device that transmitted the packet; storing, in the database, the RDA in association with the RWDU; detecting, based on the stored RDA, one or more indications of stress; and determining, based on the one or more indications of stress, an emotional state of the RWDU.

The real time responsive server method may further comprise: displaying, on a display coupled to an external electronic computing device, a graphical user interface (GUI); monitoring, by the electronic computing device, user interactions with a button displayed on the GUI; while monitoring the user interactions with the button, detecting a user interaction with the button; and based on the detecting of the user interaction with the button, determining a mood of the user.

According to an aspect of the disclosure, a method may be performed by a wearable electronic ring device, the method comprising: controlling a wearable ring display to display a first color corresponding to a first mood or emotion at a first point in time; and controlling a wearable ring display to display a second color corresponding to a second mood or emotion at a second point in time, wherein the first color is different than the second color, wherein the displayed ring color is associated with a current feeling or mood.

According to an aspect of the disclosure, a non-transitory computer-readable medium may be provided that comprises computer-executable instructions that, when executed by processing circuitry, cause the processing circuitry to perform the real time responsive server method and/or the wearable device method.

### BRIEF DESCRIPTION OF DRAWINGS

The above and other aspects, features, and advantages of certain embodiments of the disclosure will be more apparent from the following description taken in conjunction with the accompanying drawings, in which:

FIG. 1A illustrates a diagram of a networked computer system that may execute a method, according to an embodiment, according to an embodiment;

FIG. 1B illustrates a detailed view of an electronic computing device that can be used to implement the various components and/or methods described herein, according to certain embodiments;

FIG. 1C illustrates a detailed view of a responsive server that can be used to implement the various components and/or methods described herein, according to some embodiments;

FIG. 1D illustrates a detailed bird's eye (or top) view of an electronic wearable ring device used to implement the various components and/or methods described herein, according to some embodiments;

FIGS. 2A-2M2 illustrate various embodiments of the wearable electronic ring device of FIG. 1D, according to certain embodiments;

FIGS. 3A and 3B each illustrate a flow chart of a real time responsive server method 300 performed by processing circuitry, according to certain embodiments;

FIG. 4A illustrates a flow chart of a real time responsive server method 400 performed by processing circuitry, according to certain embodiments;

FIG. 4B illustrates a flow chart of a real time responsive server method 400 performed by processing circuitry, according to certain embodiments;

FIG. 5 illustrates an electronic display device displaying a mood ring application login GUI on an electronic display screen, according to an embodiment;

FIG. 6A illustrates an electronic display device displaying an example of a mood ring home GUI on an electronic display screen, according to an embodiment;

FIG. 6B illustrates a home screen GUI in a “people” mode, according to an embodiment;

FIGS. 7A and 7B illustrate a categorized search interface(s), according to an embodiment(s);

FIG. 7C illustrates a GUI, according to an embodiment;

FIG. 8A illustrates an input screen 810 for entering a mood input, according to an embodiment, according to an embodiment;

FIG. 8B illustrates a GUI including a finalized individualized view of a mood entry detail screen, according to an embodiment;

FIGS. 9A and 9B illustrate pop-up confirmation screens that may be displayed overlaying one or more GUI screens, according to an embodiment;

FIG. 10A illustrates a mood-related input history screen, according to an embodiment;

FIG. 10B illustrates a journey input history screen, according to an embodiment;

FIG. 11A illustrates an example dashboard daily GUI 1110, according to an embodiment;

FIG. 11B illustrates an insights GUI, according to an embodiment;

FIGS. 11C1 and 11C2 illustrate a planner or calendar GUI, according to an embodiment(s);

FIGS. 11D1 and 11D2 illustrate a journal GUI, according to an embodiment(s);

FIG. 11E illustrates a wellness GUI, according to an embodiment;

FIG. 12A illustrates a communities & events GUI, according to an embodiment;

FIG. 12B illustrates a type of text insight according to an embodiment;

FIGS. 13A-13C illustrate a social connections user interface/user experience (UI/UX), according to an embodiment;

FIG. 14A illustrates a data table that may be a color correspondence table, according to an embodiment;

FIG. 14B illustrates a data table including user predictive commands, according to an embodiment;

FIG. 14C illustrates a data table including detected fidget transaction data, according to an embodiment;

FIG. 14D illustrates a data table, which may be stored in the server's database, according to an embodiment;

FIG. 14E illustrates a data table showing dual correspondence, according to an embodiment;

FIGS. 15A and 15B show different versions of profile page GUIs; and

FIG. 16 illustrates App Notifications/Alerts groups, according to an embodiment.

### DETAILED DESCRIPTION OF THE INVENTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness. The terminology used herein is for the purpose of referring to specific embodiments only and is not intended to limit the invention.

With reference to the accompanying drawings, the embodiments of the disclosure will be described in detail so that those of ordinary skill in the art can easily carry out the embodiments. However, the inventive concepts may be embodied in several different forms and are not limited to the embodiments described herein.

As used herein, the singular forms “a,” “an,” and “the” include the plural forms unless the context clearly indicates otherwise. Thus, for example, reference to “an element” includes reference to one or more of such elements. The meaning of “comprising,” as used herein, specifies a particular characteristic, region, integer, step, operation, element and/or component, and other specific characteristic, region, integer, step, operation, element, component, and/or group. It does not exclude the existence or addition of anything.

### Wearable Ring System

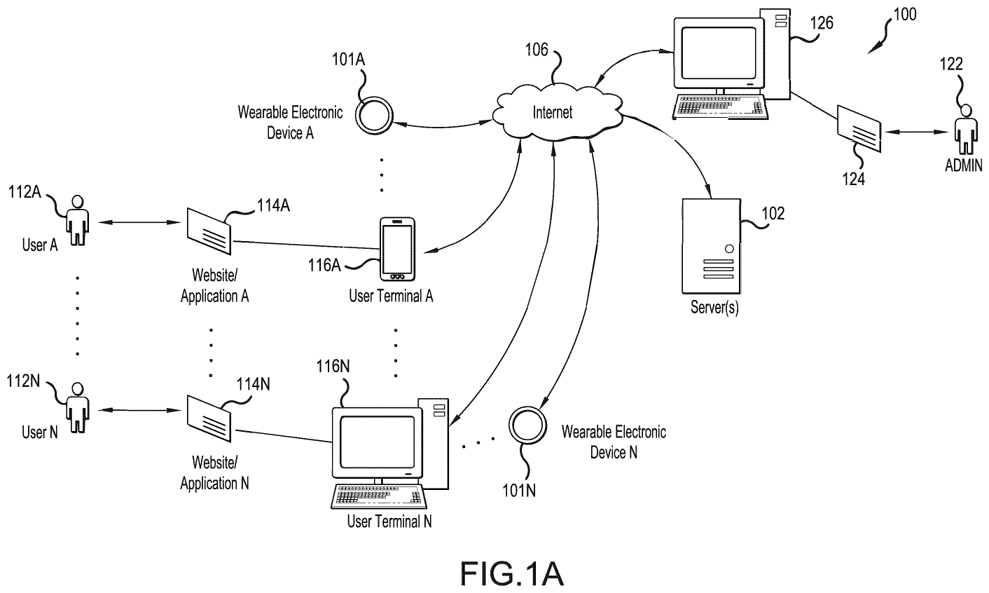

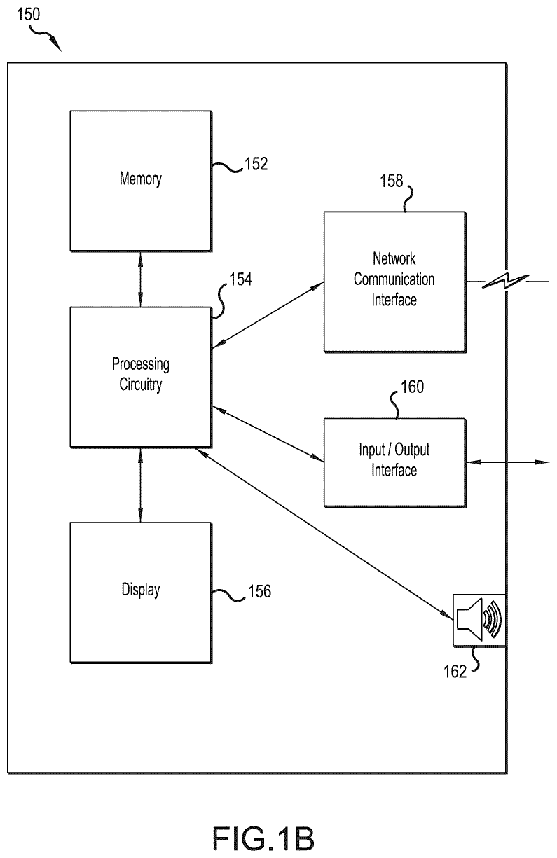

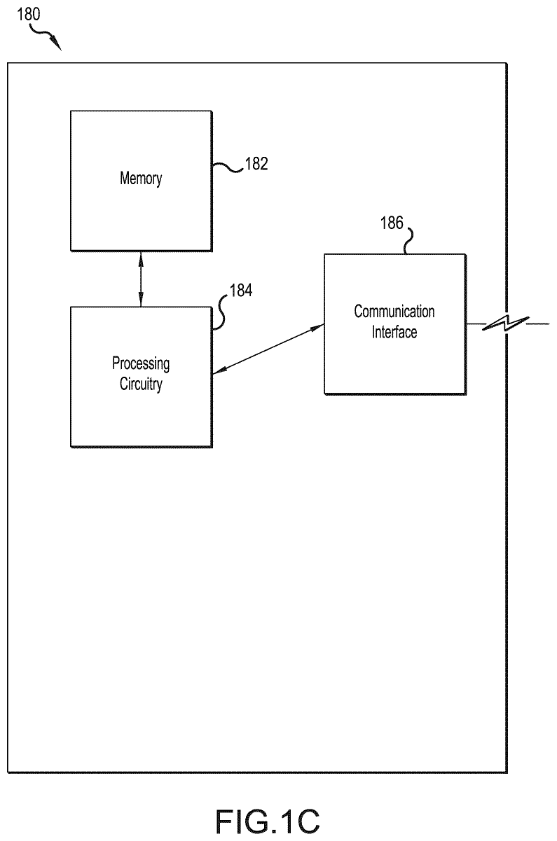

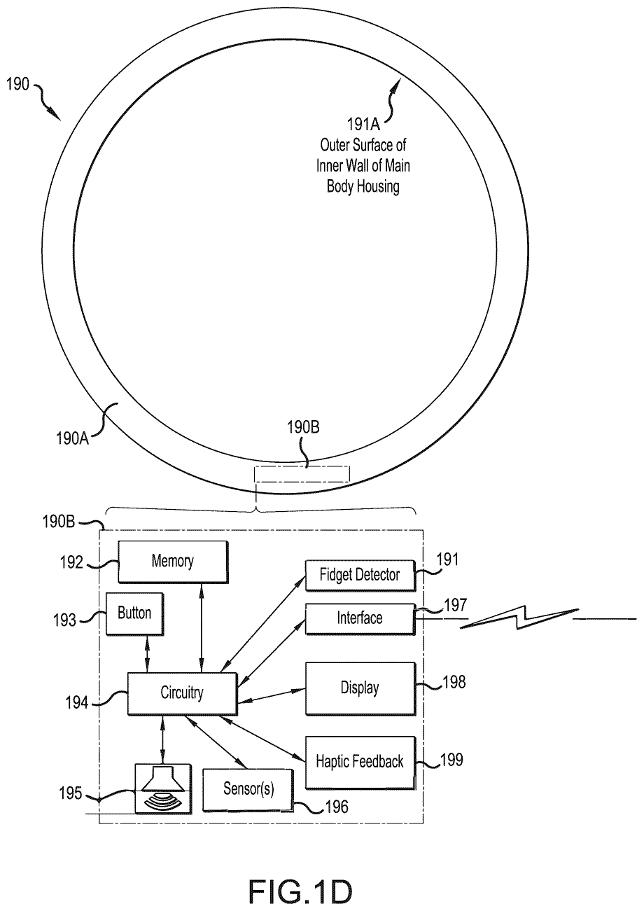

FIG. 1A illustrates a diagram of a networked computer system 100 that may execute a method according to certain embodiments. The networked computer system 100 may be a wearable health monitoring, notification, and recommendation system 100 for wearable electronic devices, such as an interactive wearable ring electronic display device. The networked computer system 100 may include one or more responsive servers (e.g., server 102) one or more wearable interactive electronic display devices (e.g., wearable ring devices 101A-101N), and a plurality of user terminals (e.g., User Terminal A (116A)-User Terminal N (116N), Admin Terminal 126). Although three user terminals and two wearable electronic ring devices are shown, the number of user terminals and wearable ring devices may be more or less. Each user terminal device may be connected to one or more ring devices, and ring devices may also connect to one or more other electronic devices, such as with one or more of multiple user terminal devices (e.g., User Terminal 116A-User Terminal 116N) and/or a responsive server(s) 102.

The user terminals 116A-116N and 126 may be communicably connected (e.g., via a network 106, such as the Internet 106) to communicate with the responsive server(s) 102 and the electronic wearable ring devices 101A-101N. The server(s) 102 may correspond to more than one networked server, such as a social media server and a user ring account server, and/or may involve distributed computing, parallel computing, a virtual server(s), cloud computing and the like. Although the processing is discussed on specific devices, the processing may be distributed.

The user terminals 116A-116N and 126 are electronic computing devices and shown as a smartphone 116A (a mobile electronic device) and a desktop computer (with a monitor) 116N, 126, respectively, but the user terminals may be any type of electronic computing device, such as a desktop personal computer (PC), a smartphone (mobile device), a tablet computer, a laptop computer, a personal digital assistant (PDA), an e-book reader, a connected smart speaker or a listening device, and the like.

According to an embodiment, the networked computer system 100 may include a plurality of user accounts stored in a database of the server(s) 102. Each of the user accounts may be associated with one or more users 112A-112N and may each correspond to a registered wearable ring account (e.g., where a User 112A-112N logs in to access the website/applications 114A-114N via the user terminal 116A-116N). The server may also store one or more admin accounts for admin users (e.g., Admin User 122 accesses a website/app 124 via user terminal 126 to provide global updates, such as adjusting weights, or adjusting a weight adjusting algorithm, managing user accounts, etc.).

The user terminals 116A-116N and 126 may correspond to any electronic computing device that a respective user 112A-112N or 126 is logged into. In this respect, for example, when user 112A is logged into the website or application 114A, the current electronic computing device the user 112A is using is user 112A's user terminal 116A. When user 112A logs in from a different electronic computing device, that different electronic computing device is user 112A's user terminal 116A. The software is application and/or webpage/portal driven, and hence, it does not matter which electronic computing device/user terminal a user logs into, as the application is accessible from different connected computing devices. The software corresponds to and/or includes the computer-executable instructions discussed below.

Admin Terminal 126 is an electronic computing device that has an admin user logged in. An admin user is a user designated as an administrator of the user accounts for the software service (e.g., the wearable ring notification user accounts). The admin user terminal 126 may communicate with the other user terminals 116A-116N. the rings 101A-101N and/or with responsive server(s) 102 over network 106.

As described above, the user terminals and/or responsive servers may perform and execute various tasks, operations and/or methods described herein. The responsive server(s) may perform some or all the processing with relation to the methods performed by the user terminals (electronic computing devices).

### Electronic Device (User Terminal)

FIG. 1B illustrates a detailed view of an electronic computing device 150 that can be used to implement the various components and/or methods described herein, according to certain embodiments. Any of the user terminals 116A-116N and 126 (and also server 102) may correspond to electronic computing device 150.

In an embodiment of the disclosure, as shown in FIG. 1B, the electronic computing device 150 may include one or more memories 152, processing circuitry 154 (e.g., a controller, a hardware processor), a display 156, a network communication interface 158, an input/output (I/O) interface 160 and a speaker 162. The speaker 162 may be internal or external (e.g., wireless headphones speakers).

The processing circuitry 154 may include and provide functionality by way of one or more processors (e.g., a hardware processor, a virtual processor, a distributed processor, central processing units (CPUs), a specialized processor, such as a Graphics Processing Unit (GPU), and/or Application Specific Integrated Circuits (ASICs)) executing computer-executable instructions (software) embodied in one or more tangible (non-transitory), computer-readable media. The computer-readable media may correspond to one or more memories 152, which may hereinafter be referred to as a memory 152. According to an embodiment, memory 152 may be a local memory 152 or local data storage 152. According to an embodiment, the memory 152 may include one or more of: media, a storage device, memory devices or chips, internal mass storage (e.g., Read-Only Memory (ROM), a Random-Access Memory (RAM)), a Compact Disc (CD), a Digital Versatile Disc (DVD), a thumb-drive, a removable hard drive, a solid-state drive, legacy magnetic media (e.g., a tape drive, a floppy disc drive), a specialized ASIC-based device, and/or the like.

The software implementing various embodiments of the present disclosure can be stored in the memory (e.g., storage devices and/or media) and executed by processing circuitry 154. “Computer readable media” or “computer readable medium” as used in connection with the disclosure include non-transitory media, transmission media, and/or carrier waves.

The computer readable media and/or memory 152 may store computer code/instructions (software) for performing various computer-implemented functions. The computer code may include instructions that, when executed by the processing circuitry 154, causes the processing circuitry to execute various functions, processes, parts, operations and/or methods (collectively referred to hereinafter as functions or operations). Reference to a computer-readable media can include a circuit (such as an integrated circuit (IC)) storing software for execution, a circuit embodying logic for execution, or both, where appropriate. The present disclosure includes any suitable combination of hardware and software.

The various functions, operations, and methods pertinent to this disclosure are explained in detail herein. The processing circuitry 154 may be configured to perform the various operations (e.g., by being programmed to do so via computer executable instructions) discussed in more detail herein.

The electronic computing device 150 may further include a communication interface 158 electrically coupled to the processing circuitry 154. The communication interface 158 may be configured to receive data transmitted from one or more electronic end user devices (e.g., user terminal devices, the one or more electronic ring devices, and/or other electronic devices) and/or one or more servers. The communication interface 158 may include a network communication interface 158. The communication interface 158 may be configured to transmit, via, for example, control by the processing circuitry 154, data to the one or more electronic computing devices over a network (wired or wirelessly), such as, via an interface to one or more communication networks (e.g., via the communication interface 158). The processing circuitry 154 may be configured to control (or cause) the display 156 to display information, or alternatively, transmit the to-be-displayed information over the communication interface 158, or over the input/output interface 160 to another electronic computing device (e.g., smartphone, computer monitor) for external display.

As shown in FIG. 1B, the electronic computing device 150 may also include or be at least configured to be electrically connected (or wirelessly connected) to one or more electronic input/output (I/O) devices, including peripheral output devices (e.g., external, or internal). The electronic computing device 150 may use the input/output (I/O) interface 160 to communicate with one or more external peripheral devices, such as, one or more of: a display device (e.g., an external desktop monitor, a touch screen display panel, etc.), a keyboard, a microphone (e.g., for voice input), a camera (e.g., for gesture input), a mouse, a speaker, a microphone, a joystick, a scanner, a trackpad, a printer, a sensor, and/or a biometric capture device (e.g., a device that captures one or more of: fingerprint, palm print, iris information, etc.). The peripheral devices may include user input devices (e.g., a mouse, keyboard, microphone) and/or output devices (e.g., speaker, display) and may be connected to the processing circuitry 154 by the I/O interface 160 or the network communication interface 158. The electronic display device may correspond to a smartphone and/or electronic computing device and include a touch-screen display device that is configured to detect an interaction (e.g., touch, tap or long or short press) with the touchscreen, and identify information (e.g., position on the display screen) regarding the interaction. The GUIs may be used to prompt for and/or receive user input, and also provide virtual buttons for navigation to multiple different GUI screens related to the wearable ring device. The information displayed by the GUIs may include touch menu buttons for navigating to sub-menus.

Throughout the drawings, it should be noted that like reference numbers are used to depict the same or similar elements, features, and structures.

The electronic display device may correspond to and/or be electronically coupled to the electronic computing device, the wearable ring device and/or a server.

The I/O interface 160 may be wired (e.g., universal serial bus (USB) drive, external RAM/ROM, external processor, data port) or wireless (e.g., short-range communication protocols, syncing, Bluetooth®, radio frequency (RF), near field communication (NFC) or Wi-Fi®). The I/O interface 160 may be electrically coupled to the processing circuitry 154 and the processing circuitry 154 may be configured to control (or cause) the I/O interface 160 to transmit information between the device 150 and other electronic devices or peripherals. At least a portion of the network communication interface 158 and or the I/O interface 160 may be provided on an outer surface of a housing of an electronic computing/display device (e.g., a smartphone). The network communication interface 158 and or the I/O interface 160 may include similar and/or the same overlapping functionality.

The software may cause the processing circuitry and specifically the processor(s) to execute particular processes or particular parts of particular processes described herein, including defining data structures stored in RAM and modifying data structures according to the processes defined by the software. In addition, or as an alternative, the processing circuitry may provide functionality because of logic hardwired or otherwise embodied in a circuit, which can operate in place of or together with software to execute particular processes or particular parts of particular processes described herein. Reference to software can encompass logic, and vice versa, where appropriate.

Networks may be, for example, wired, wireless, or optical. Networks can provide real-time data transfer and include a local area network (LAN), a wide-area network (WAN), and/or the Internet. Examples of networks include Ethernet, wireless LANs, cellular networks (e.g., Global System for Mobile Communications (GSM)), 3G, 4G, 5G, Long-Term Evolution (LTE).

The display 156 may include any type of display including a projector or a display screen, such as, a touch screen display panel, an organic light-emitting diode (OLED) screen, a liquid crystal display (LCD) screen, a cathode ray tube (CRT) screen, and/or augmented reality glasses. The display 156 may be electrically coupled to the processing circuitry 154 and the processing circuitry 154 may be configured to control (or cause) the display 156 to display information (e.g., display the information discussed in the methods and operations below). The display 156 may be provided on an outer surface of a housing of an electronic computing/display device (e.g., a smartphone) or be an external display monitor connected to a standalone electronic computing device (e.g., a PC connected to a monitor, where the monitor is the display).

The speaker(s) 162 may include any type of speaker including a smartphone/tablet speaker(s) and/or wired or wireless headphone speakers (also referred to as earbuds). The speaker(s) 162 may be electrically coupled to the processing circuitry 154 and the processing circuitry 154 may be configured to control (or cause) the speaker(s) 162 to output sound, such as a notification. The speaker(s) 162 may be provided on an outer surface of a housing of an electronic display device (e.g., a smartphone) or be an external speaker. The speaker may include a microphone, or a microphone may be provided separately to record sound data, such as speech data.

### Real Time Responsive Server(s)

FIG. 1C illustrates a detailed view of a responsive server 180 that can be used to implement the various components and/or methods described herein, according to an embodiment(s). The responsive server(s) 102 in FIG. 1A may correspond to the responsive server 180 illustrated in FIG. 1C.

In an embodiment of the disclosure, as shown in FIG. 1C, the responsive server 180 may include one or more memories 182 (e.g., a local memory or local data storage), processing circuitry 184 (e.g., a controller, a hardware processor) and a communication interface 180 electrically coupled to the processing circuitry 184. The communication interface 186 may be configured to receive data transmitted from one or more external electronic computing devices (e.g., electronic computing devices 116A-116N, 126, wearable devices), and/or one or more other servers.

The processing circuitry 184 may be configured to store, in memory 182, data received from various sources over the network. For example, processing circuitry 184 may be configured to store information received as data received from the communication interface 186.

The responsive server 180 may be configured to provide automatic real time updates to the respective electronic computing devices, as discussed below. Real time may correspond to instantaneous, near real time (near instantaneous) or a combination thereof. The processing circuitry 184 may be connected to each of the other components of the server(s) 180 and may communicate, via communication interface 186, with one or more of the components of the electronic device 150 of FIG. 1B. The communication interface 186 may correspond to and include the same functionality as network communication interface 158 and/or I/O interface 160 discussed above.

### Electronic Wearable Ring Device(s)

FIG. 1D illustrates a detailed bird's eye (or top) view of an electronic wearable ring device 190 used to implement the various components and/or methods described herein, according to some embodiment(s). For example, the wearable electronic ring device 190 in FIG. 1D may correspond to the wearable electronic ring devices 101A-101N in FIG. 1A. The ring device may be a wearable health, fitness and mood tracking device that includes a processor or circuit that executes instructions that cause the processor or circuit to perform operations.

The wearable ring device may be a “smart” electronic health tracking device that provides therapy through sensory engagement via visual and tactile reinforcement while also connecting via Bluetooth® to software enabling data transfer, remote monitoring, and interactive programming. Features of the device include, it is a ring worn on the index, middle or ring fingers of the user's non-dominant hand with a “fidget” wheel that enables customizable color changing to express different emotions/feelings.
1. Mood input—journaling about how you feel about something in real time;
2. Other users connect and see your emotion and then connect with user to encourage, compliment, etc.
3. User associates color with emotion. For example, if they like yellow they may say yellow is happy.
4. App can suggest activities and people that you had an experience of memory with; your feedback controls content and experiences. The prior art is silent regarding spinning/fidgeting controlling the color on the ring.

In an embodiment of the disclosure, as shown in FIG. 1D, the wearable electronic ring device may include a wearable ring body housing 190A and electronic wearable ring components 190B. FIG. 1D shows an exploded view of the wearable ring components and the broken line indicates that the electronic components 190B may be located within and/or on a surface of the ring body housing 190A.

The wearable ring body housing 190A may have a cylindrical shape as shown in FIG. 1D, and an inner wall having an outer surface 191A configured to engage with a digit of a dactyl mammal as a wearable ring. The outer surface 191A may be firmly attachable to the digit (e.g., finger) of the dactyl mammal as the wearable ring. Although a wearable ring device is shown, according to an embodiment, the wearable electronic device 101A-101N and/or 190 may be another type of electronic wearable device, such as a smartwatch.

The ring device may be a wearable wellness device providing personal insights about mood using emotional expression and ring colors. It uses haptics and sounds to notify users about a notification from the app. Ring has swiping, tapping, and clicking functionality. Ring has a tiny screen to show the emotional expression chosen in the App. MUUD input can also be changed directly from the ring. MUUD App Notification/Functionality: Web and mobile application designed to empower people by facilitating personal and community wellness activities. Offers: Community networking, Habit tracking, Mindfulness training. Functionality: User can choose an emotion and his/her favorite color associated with that emotion and share them with his/her community. MUUD ring reflect the MUUD input from the app (emotion and color).

Making connection with community of people using the app in the neighborhood and also other social platforms (e.g., Instagram, TikTok).

Positive reinforcement device which is a complement to the app and can also remotely control some of the App's functionality. MUUD Ring has the following potential functionalities (All of these functionalities can be changed in the app).

Ring main functionalities: Alarm feature which can be used for key notifications (related to either new or existing wellness activity). It should be a reminder tool. For updates related to friends and connections. It should be a reminder tool. For updates related to friends and connections. Sending notifications regarding inactivity in order to keep the user up to date. Milestone tracker (when milestone happens ring can alert).

Taps: Once: Immediately responds to some notifications or waking up from sleep mode. Twice: Engage. Press click: initiating an action. Press hold: Emergency (fast flash of light-5 or more seconds). Turns: Could be a stress relief tool which can also return the total number of turns. Swipes: Lateral swipe: respond to invitation from the app or new journey or invitation from connection (accept or reject). Ventral swipe. Flashes: Could be used to inform the user about an action happened on the ring (Haptics and flashes are more for inner circles). Haptics: Same thing as flashes but for more serious notifications.

Goal: If a change of mood happens in a user, That can be input to the app and the user's community will be aware of his/her mood currently, then the user can take journeys with people in his/her community or take wellness journeys inside the app which can help improving the user's mood.

A journey starts by receiving a flash on the ring and can end with a haptic

### Wearable Ring Electronic Components

The Electronic Wearable Ring Device(s) (also referred to as a ring) may be a conduit to an application or app to facilitate personal and community wellness activities. The ring may be a positive reinforcement tool that allows user to interact with the mood/MUUD app seamlessly and keeps the user connected to his/her inner circle.

The wearable ring electronic components 190B may include components including a fidget detector 191, one or more wearable ring memories 192 (e.g., a local memory or local data storage), one or more button(s) or hard key(s) 193, wearable ring processing circuitry 194 (e.g., a controller, a hardware processor), a speaker 195, one or more sensor(s) 196, a wearable ring interface 197, a wearable ring display 198 and a haptic feedback mechanism 199. Each of the components may be electrically coupled to the wearable ring processing circuitry 194. The haptic feedback mechanism 199 may include a vibrator and/or piezoelectric element that enable the ring to communicate (e.g., provide an alert/notification) to the wearer without color, such as a notification or mood booster (e.g., after a long period in a low mood state).

The fidget detector 191 may correspond to hardware and/or software functionality that detects and records a fidget event which may correspond to a user performing a click or button press of a fidget detector of a wearable device, such as a wearable ring device. Other examples of fidget detection may include detecting a finger tap, detecting movement of a sliding element in a first direction from a first position to a second position and/or detecting movement in a second direction opposite the first direction, and/or detecting movement of a spherical ball attached in a socket of a mobile ring device.

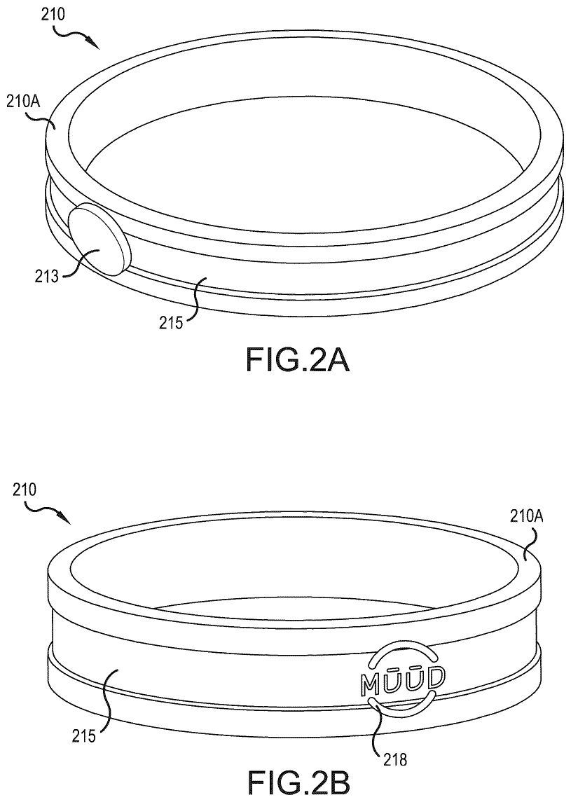

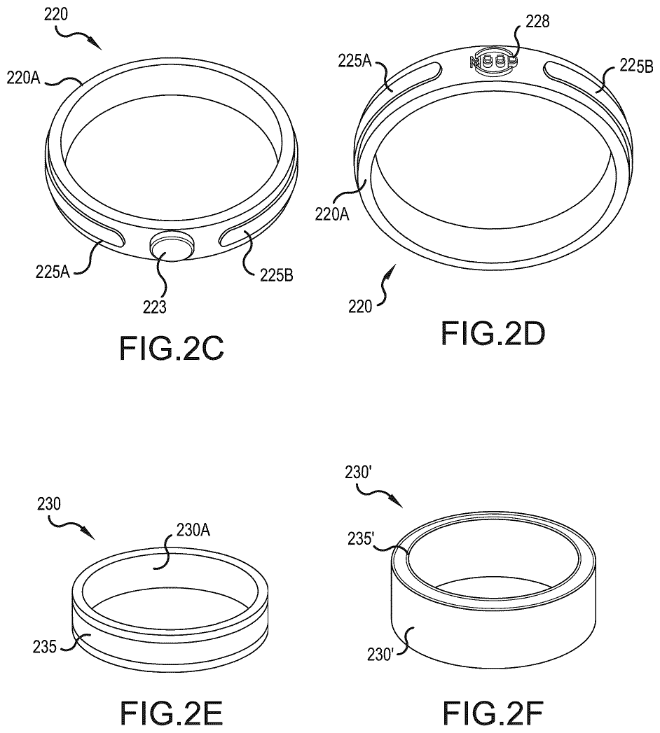

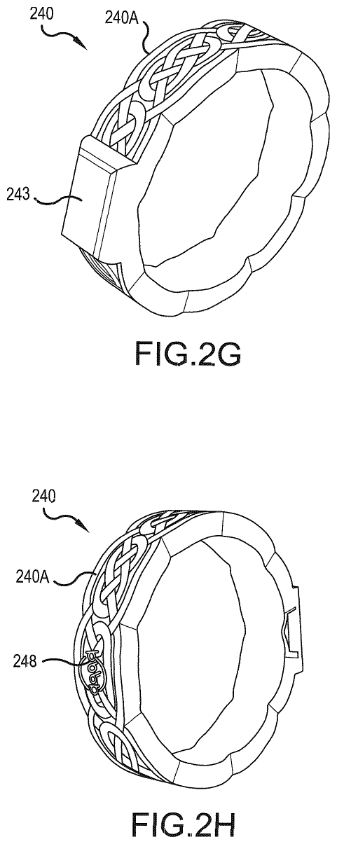

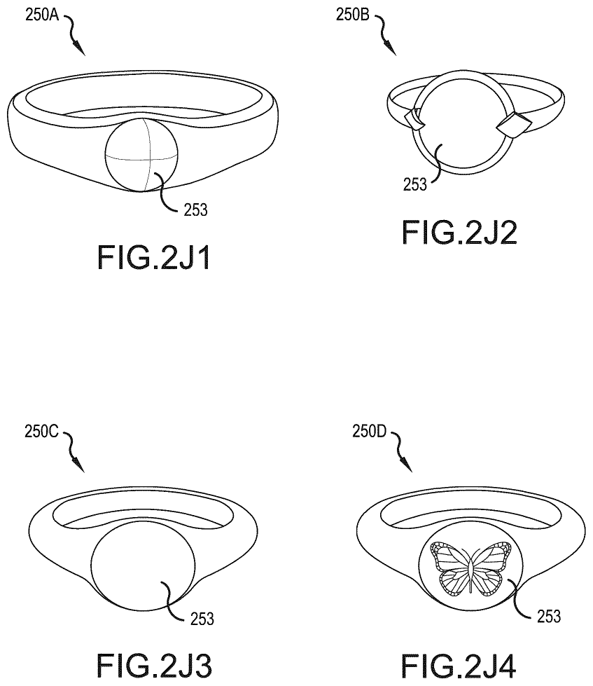

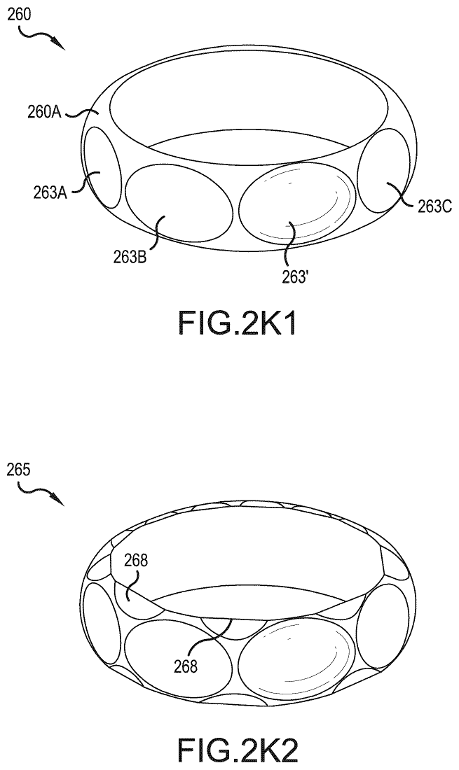

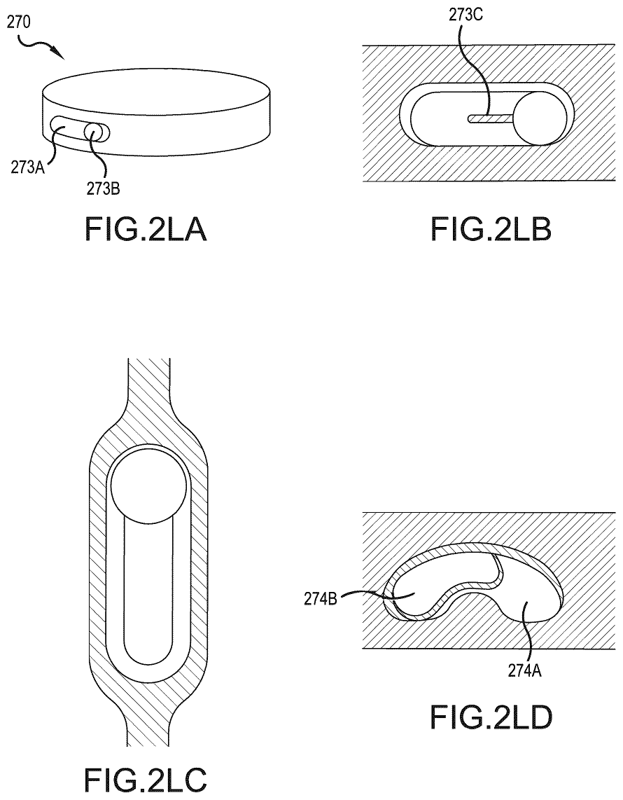

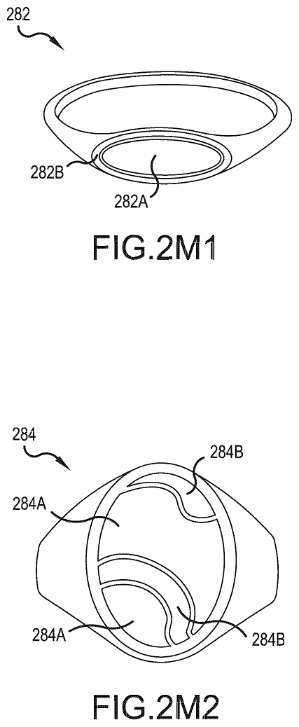

The memory 192 may include a database storing wearable ring events and program/software instructions that are executed by the processing circuitry 194. The button(s) 193 may correspond to one or two or more buttons 193. The buttons 193 may be hard keys, as shown in, for example, FIGS. 2A-2H. However, the button(s) 193 may be soft keys (e.g., when the display is a touch sensitive display) and/or the button may correspond to capacitive touch panel, which may or may not include a display backscreen.

The sensor(s) 196 may correspond to one or more sensors, which may be provided inside the ring body housing or on a surface of the ring body housing. The sensor (196) are discussed in more detail with relation to passive data collection below.

The interface 197 may be a communication interface may be configured to receive data transmitted from one or more external electronic computing devices (e.g., electronic computing devices 116A-116N, 126), and/or one or more other servers. The processing circuitry 194 may be configured to store, in the memory 192, data received from various sources over the network. For example, the processing circuitry 194 may be configured to store information received as data received from the communication interface 197. The interface 197 may be configured to receive automatic real time information from the server, such as updates and notifications from the server. The processing circuitry 194 may be connected to one or more or each of the other components of the server(s) 180 and may communicate, via communication interface 197, with one or more of the components of the electronic device 150 of FIG. 1B. The communication interface 197 may correspond to and include the same functionality as network communication interface 158 and/or I/O interface 160 discussed above.

The wearable ring display 198 may include any type of display including, for example, a display screen, such as, a touch screen display panel, a light-emitting diode (LED) strip or screen, or a liquid crystal display (LCD) screen. The wearable ring display 198 may be electrically coupled to the processing circuitry 194 and the processing circuitry 194 may be configured to control (or cause) the display 198 to display information (e.g., display the information discussed in the methods and operations below). The display 198 may be provided on an outer surface of the wearable ring housing 190A of the wearable electronic ring device 190.

The ring may have a dimming feature that enables energy conservation as well as security (stealth mode). There are 6 options: a) full light/haptics b) full light only c) haptics only d) dim light only e) stealth mode (no light/no haptics) f) hybrid mode (personal alerts only versus community alerts only).

Lights and Haptics could be used to inform the user about an action happening on the ring (Haptics and flashes are more for inner circles). Single haptic is used for acknowledgment of the app interaction. Double haptics is used for notifications. Triple haptics is used as an emergency alert.

Any of the user terminals 116A-116N and 126 may correspond to electronic computing device 150. FIGS. 1B and 1D illustrate various components that may be included in any of the electronic computing device 150 illustrated in FIG. 1B, the responsive server 180 illustrated in FIG. 1C, and and/or the wearable electronic device 190 of FIG. 1D.

According to various embodiments, the housing 190A, display 198 and button 193 of the wearable ring device 190 may have various embodiments as shown in FIGS. 2A-2M2. Embodiments of the wearable ring device 190 (including one or more of the outer surfaces of the wearable ring housing 190A, and wearable display 198) are shown in FIGS. 2A-2M2, and each may have the structure, including some or all of the electronic components (on the inside) from FIG. 1D, according to certain embodiments.

The detailed views illustrate various components that may be included in the electronic computing device 150 illustrated in FIG. 1B, the responsive server 180 illustrated in FIG. 1C, and/or the wearable ring device 190 of FIG. 1C. The components shown in FIGS. 1A-1D are exemplary in nature and are not intended to suggest any limitation as to the scope of the disclosure. While explaining the various electronic devices according to certain embodiments, a detailed description of the contents (e.g., processing circuitry, media, memory, communication interface) overlapping with previously described electronic computing device are omitted.

According to an embodiment, the structure and/or functionality of the electronic computing device, the responsive server and/or wearable electronic device may be included within the same device or performed/provided for by another electronic device. The functionality of the server/devices, as defined by the logged in user, may also be performed by one of the other user terminal electronic computing devices.

In an embodiment, the user terminal(s), server and/or wearable device may perform the method 300, the method 350, the method 400, and/or the method 450. Portions of the method 300, the method 350, the method 400, and/or the method 450 may overlap and/or be combined into one method.

### Electronic Wearable Ring Device Types

The electronic wearable ring device may correspond to any of the wearable ring device types in the embodiments of FIGS. 2A-2M2. FIGS. 2A-2M2 illustrate various embodiments of the wearable electronic ring device 190 of FIG. 1D. Some embodiments may not include all of the elements of FIG. 1D.

### User Interface

The MUUD ring (e.g., a wearable electronic ring device) will feature a touch-sensitive button that, when pressed, will respond to the certain notifications coming from the app. The display will be a small, circular OLED screen that will show emotional expression and potentially other info (optional). MUUD ring will also have a built-in microphone and speaker for recording

### System Interface

Firmware Update: The ring will have the capability to receive firmware updates through a connected smartphone or PC.

Connectivity: MUUD ring will connect to the user's smartphone via Bluetooth 5.0 for data and control functionality.

Power: The ring will be powered by a rechargeable battery and will have a battery life of at least 5 days under normal usage.

Storage: The ring will have at least 4 GB of storage for storing data related to its corresponding app.

Compatibility: The ring will be compatible with both iOS and Android smartphones.

### Sustainability

Operating Temperature: MUUD ring will be able to operate within a temperature range of −20° C. to 50° C.

Storage Temperature: MUUD ring should be stored in temperatures ranging from −30° C. to 70° C.

Humidity: MUUD ring will be able to operate within a humidity range of 10% to 90% non-condensing.

Water Resistance: MUUD ring will be water-resistant up to a depth of 3 meters for 30 minutes.

Shock and Vibration: MUUD ring will be able to withstand vibration and shock in accordance with IEC 60068-2-6 and IEC 60068-2-27.

Altitude: MUUD ring will be able to operate at altitudes up to 15,000 (10,000) feet.

EMI/EMC: MUUD ring will comply with the EMI/EMC requirements of the FCC and CE.

Battery Life: MUUD ring will have a battery life of at least 5 days under normal usage.

Recycling: MUUD rings will be designed to be recyclable and made of eco-friendly materials.

Packaging: MUUD rings will be packaged in a recyclable and eco-friendly material.

### Reliability

MTBF (Mean Time Between Failures): MUUD ring will have an MTBF of at least 5 years.

MTTR (Mean Time To Repair): MUUD ring will have an MTTR of less than 2 hours.

Failure Rate: MUUD ring will have a failure rate of less than 0.1%.

Lifetime: MUUD ring will have a lifetime of at least 5 years.

Durability: MUUD ring will be able to withstand at least 50,000 button presses and 10,000 charge cycles.

Water-resistance: MUUD ring will be water-resistant up to a depth of 3 meters for 30 minutes.

Quality Control: The ring will undergo rigorous testing and quality control to ensure that it meets the reliability requirements.

### Physical

Size and Shape: MUUD ring will be designed to fit comfortably on the finger and will have a diameter of no more than 25 mm and a thickness of no more than 10 mm.

Material: MUUD ring will be made of durable and lightweight materials, such as titanium or stainless steel.

Finish: The ring will have a polished or brushed finish.

Color: The ring will be available in multiple colors, such as silver, black, and gold.

Weight: The ring will have a weight of no more than 20 grams.

Packaging: The ring will be packaged in a box that includes the ring, a charging cable, and a user manual.

Battery life: The ring will have a battery life of at least 5 days under normal usage.

Battery Charging: The ring will be charged via a USB cable and will have a charging time of no more than 2 hours.

Battery Indicator: The ring will have a battery indicator that shows the remaining battery life.

### Compliance (Federal Vs State Law)

FCC Compliance: MUUD ring will comply with the Federal Communications Commission (FCC) regulations for radio frequency devices.

CE Compliance: MUUD ring will comply with the European Union (EU) regulations for electromagnetic compatibility (EMC) and low voltage equipment.

RoHS Compliance: MUUD ring will comply with the Restriction of Hazardous Substances (RoHS) Directive and will not contain any hazardous materials.

REACH Compliance: MUUD ring will comply with the EU Regulation on Registration, Evaluation, Authorization, and Restriction of Chemicals (REACH) and will not contain any substances of very high concern (SVHC).

WEEE Compliance: MUUD ring will comply with the EU Waste Electrical and Electronic Equipment (WEEE) Directive and will be designed

### Security

Software Updates: MUUD ring will have the capability to receive software updates to ensure that the device is running the latest security patches and updates.

Malware Protection: MUUD ring will have malware protection to detect and prevent malware from infecting the device.

Remote Wipe: MUUD ring will have the ability to remotely wipe all data in the event of loss or theft.

Encryption: All data stored on and transmitted by the ring will be encrypted to protect personal information and prevent unauthorized access.

Storage: MUUD ring has a separate case which stores the data more securely and therefore less susceptible to data theft.

Compliance: MUUD ring will comply with relevant security standards and regulations, such as ISO 27001 and SOC 2.

Cost & Pricing

### Quality

Quality Assurance Plan: A comprehensive Quality Assurance (QA) plan will be developed and implemented to ensure the quality of the MUUD ring.

Design for Manufacturability: MUUD ring will be designed for manufacturability to ensure that it can be produced consistently and with high quality.

Design for Reliability: MUUD ring will be designed for reliability to ensure that it has a low failure rate and meets the customer's needs and expectations.

Design for Testability: MUUD ring will be designed for testability to ensure that it can be easily tested and diagnosed in the event of a problem.

Testing Requirements: Testing requirements will be defined and documented to ensure that the ring meets the customer's needs and expectations.

Test Equipment: Test equipment will be purchased or rented to ensure that the MUUD ring can be tested and diagnosed accurately and efficiently.

Test Procedures: Detailed test procedures will be developed and documented to ensure that the ring is tested consistently and accurately.

Inspection Requirements: Inspection requirements will be defined and documented to ensure that the MUUD ring is inspected consistently and accurately.

Inspection Equipment: Inspection equipment will be purchased or rented to ensure that the MUUD ring can be inspected accurately and efficiently.

Inspection Procedures: Detailed inspection procedures will be developed and documented to ensure that the MUUD ring is inspected consistently and accurately.

### Manufacturing

Manufacturing Process: A detailed manufacturing process will be developed and documented to ensure that the MUUD ring is produced consistently and with high quality.

Process Control: A process control plan will be developed to ensure that the manufacturing process is controlled and that the MUUD ring is produced consistently and with high quality.

Product Traceability: Product traceability will be implemented to ensure that the ring can be traced from raw material to final product.

### Support & Serviceability

Ring can be thought of as a stylish wearable rather than a tech device (similar to a piece of a jewelry). Ring is a tool that can facilitate connections.

Warranty: A warranty will be provided to the customer to ensure that the MUUD ring is covered against defects and failures.

Technical Support: Technical support will be provided to the customer to ensure that the MUUD ring is supported efficiently.

Training: Training will be provided to the customer to ensure that the MUUD ring can be used and maintained effectively and efficiently.

Documentation: Documentation will be provided to the customer to ensure that the MUUD ring can be used and maintained properly.

### Usability

User-friendly design: MUUD ring will be designed to be user friendly and easy to use.

Easy configuration: MUUD Ring will be easily configurable through accompanying app (MUUD App) to meet the needs of the user.

Intuitive interaction: MUUD Ring will feature an intuitive interaction design to ensure that the user can easily interact with the Ring.

Accessibility: MUUD Ring will be accessible to users with disabilities and will be designed to be usable by a wide range of users.

Feedback mechanism: MUUD Ring will provide feedback to the user to confirm.

Error Handling: MUUD Ring will be designed to handle errors gracefully and provide clear error messages to the user.

Compatibility: MUUD Ring will be compatible with a wide range of devices and platforms to ensure it can be used by a wide range of users.

The ring device provides the ability to always stay connected with inner circles.

Being able to manage invitations and notifications from inner circle by just responding to ring without having the need to go to the app.

The ring may have a basic simple design, a pro advanced design and a premium sophisticated design. The basic design may include a steel material, bluetooth, color change and notifications, functionalities inside the app, temperature sensor and meaningful usage and utility with fidget tool (measurement of anxiety). The pro design may be a titanium material, bluetooth, ring color change and emoji, temperature and oxygen sensor, pulse oximeter, oxygen sensor, fidget tool (combination of click and spinning to do certain functionalities) and contactless payment (e.g., Apple and/or Google pay). Premium may be gold material from high end jewelers, pulse oximeter, oxygen sensor, conductance (moisture measurement), contactless payments, and accelerometer. Instead of metal, enamel and ceramic materials may be used to provide different colors.

FIGS. 2A and 2B illustrate a wearable electronic ring device 210. FIG. 2A illustrates a front side (or primary action button side view) of the wearable electronic device 210 including a wearable ring action button 213, and a wearable ring curved display 215, each provided on an outer surface of a housing 210A. FIG. 2B illustrates a rear side (an opposite side as the front side) view of the wearable ring device 210 including, a second wearable ring combo display and secondary action button 218 provided on an outer surface of an outer wall of the housing 210A. FIGS. 2A and 2B may show the same ring from different views.

There may be two action buttons, located on opposite sides of the device—the button on the right-hand side depicting personal activity and left-side depicting community activity.
2) Rule of 3: The Rule of 3 states that user functions should ideally be executed in one or two steps, such as one tap, one click, or one flash, but may require up to three steps for complex functions that contain uncommon information, such as emergency data. Software functions may require a maximum of three user input steps, with the exception of complex functions that contain uncommon information, such as emergency data, which may require additional steps.

According to an embodiment, a first button on the medial side may depict personal activity and a second button on the lateral side depicting community activity (symmetric sides: left and right side). When providing a rating/review for journeys-tapping either once, twice or 3 times inputs to what extent the user liked or disliked their experience. Tapping once: Immediately responds to some notifications or waking up from sleep mode. Double tap: Reaction to certain notifications. Press Click: initiating an action (MUUD Input).

When in standby mode, tapping twice on each side would either go up or down the list of MUUD inputs (color and emoji).

FIGS. 2C and 2D illustrate a wearable electronic ring device 220, according to an embodiment. FIG. 2C illustrates a front (or primary button side) view of the wearable electronic ring device 220 including a primary wearable ring button 223, and two curved displays 225A and 225B (which may be semi-circle shaped), each provided on an outer surface of an outer wall of a housing 220A. FIG. 2D illustrates a rear (or opposite side as the front side) view of the wearable ring device 220 including, a secondary wearable ring display 228 provided on an outer surface of an outer wall of the housing 220A (the secondary wearable ring display 228 may technically be the third display after 225A and 225B).

The curved display of the electronic ring device may conform to an outer circumference of the ring device either fully around the circumference (as in FIGS. 2A and 2B) or partially or almost fully but not fully around (as in FIGS. 2D and 2E). In addition, the displays may include button functionality.

FIGS. 2A and 2B may show the same ring from different views and/or show two different rings, according to an embodiment. The wearable ring device 230′ may include a similar configuration as wearable ring device 230 but wearable ring device 230′ may include two displays 225A, 225B having shorter display lengths compared to display 215 such that the ring device 230′ has additional housing space between the primary button and the display(s).

The display may be LED strips. In addition, the displays may include button functionality. The front side may correspond to a side facing a palm when the ring device is worn and the rear side may correspond to a side facing in the same direction as knuckles when the ring device is being worn, according to an embodiment.

The secondary wearable ring buttons 218 and 228 may include a design, word, or trademark (e.g., shown as the word “MOOD”). The wearable ring displays 215, 225A and 225B may be LED strips (as shown) or tiny or miniature display screens.

FIGS. 2E and 2F illustrate a wearable ring device 230, 230′ including a wearable ring display 235, 235′ provided on an outer surface of a housing 230A, 203′. The view of FIGS. 2E and 2F may correspond to a front and/or rear view of the wearable ring device 230, 230′. That is, the wearable ring device 230, 230′ may include a display 235, 235′ that continues seamlessly around the circumference of the ring device 230, 230′. The display may be touch sensitive and act as a button or fidget detector. The difference between FIGS. 2E and 2F is that the display 235 is provided on an outer surface opposite the outer surface of the inner wall that attaches/detaches to the digit, whereas the display 230′ is shown as provided on a surface perpendicular to the outer surface of the inner wall that attaches/detaches to the digit. Both displays may be integrated seamlessly with standard ring dimensions, and the curved display may be provided on different surfaces of the wearable ring device.

FIGS. 2G and 2H illustrate a wearable ring device 240. FIG. 2G illustrates a front side (or primary button) view of the wearable ring device 240 including a wearable ring button 243 provided on an outer surface housing portion 240A. FIG. 2H illustrates a rear side view of the ring 240 including a secondary wearable ring display 248 provided on an outer surface housing portion 240A. The outer surface of the housing 240A of the wearable ring device 240 may be a display made of gold and may perform the same functionality as other displays described herein.

FIGS. 2J1-2J4 illustrate a wearable ring device(s) 250A, 250B, 250C and 250D, respectively. Each of the wearable ring devices in FIGS. 2J1-2J4 include a rotatable display 253 provided in a socket provided within a wearable ring housing of the wearable rings. The rotatable display 253 may incorporate display functionality but also may perform functionality related to the fidget detector and/or the button(s).

FIGS. 2K1 and 2K2 illustrate a wearable ring device 260 and 265, respectively. FIG. 2K1 illustrates a display side (or primary button) view of the wearable ring device 260 including wearable ring buttons 263A, 263B, 263C, and a display 263′, each of which are provided on an outer surface housing portion 260A.

FIG. 2K2 is based on the wearable ring device 260 but further includes a tapered edge 268 which may also detect fidgets and/or perform button functionality. The outer surface of the housing 260A of the wearable ring device 260 may be made of gold and may perform the same functionality as other displays described herein.

FIG. 2LA illustrates an embodiment of wearable ring 270 with a sliding element 273B provided in a sliding recess 273A. The sliding recess may be provided in an outer surface of a housing of a wearable ring device discussed above. The sliding recess 273A may provide a limited amount of movement of the sliding element 273B around the circumference of the ring device. The combination of the sliding element 273B and sliding recess (or groove) 273B may correspond to a button and/or a fidget detector.

FIG. 2LB shows the sliding element and sliding recess with a track 273C that provides through hole by which the sliding element may be connected to a detector (e.g., a sensor, the fidget detector and/or a button). FIG. 2LC illustrates an example of the fidget detector 273 where the width of the ring band is enlarged at an area where the fidget detector (e.g., sliding element/sliding recess) is provided. FIG. 2LD illustrates a bean shaped sliding element 274B provided within a longer bean shaped recess 274A.

FIGS. 2M1 and 2M2 each show a wearable ring device including a display including two display portions. FIG. 2M1 illustrates a ring 282 including a primary display portion 282A and a secondary display portion 282B. FIG. 2M2 illustrates a ring 284 including a primary display portion(s) 284A and a secondary display portion(s) 284B. In FIG. 2M2, the primary display portion(s) 284A and secondary display portion(s) 284B may be interleaved with each other. FIGS. 2M1 and 2M2 include rings with two distinct displays. The bigger display area may show the wearer's mood and the other, smaller display may display the mood of one or two of the closest friends in the community. In this option, there would also be a choice to have both windows display your mood.

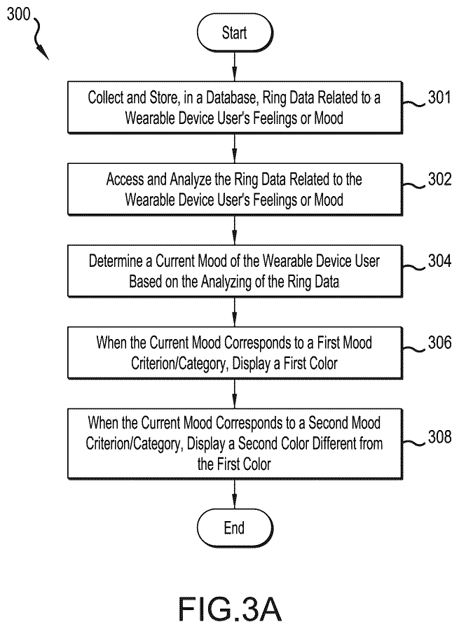

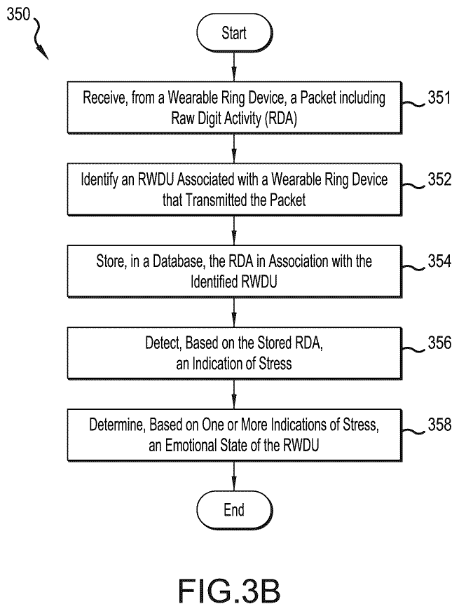

The processing circuitry may correspond to the processing circuitry 154, 184, or 194 discussed with reference to FIGS. 1B, 1C, and 1D, respectively above, or a combination thereof, such as a combination of the processing circuitry of the electronic computing device 150 being controlled by (or receiving user interface information from) the responsive server 180, and/or the wearable electronic device receiving notification/alert information from the electronic computing device and/or responsive server. As discussed herein, the processing circuitry may be configured to execute code (instructions code) stored in a memory, which causes the processing circuitry to execute a method, including one provided by a website and/or an application. The real time responsive server method 300 executed by the processing circuitry (e.g., one or more of 154, 184 and/or 194) may include Operations 301-308, as shown in FIG. 3A, according to an embodiment. Although the operations 301-310 are shown in an order in FIG. 3A, the Operations 301-310 may be performed in a different order. In addition, other methods and operations discussed herein may be performed in various orders of operation.

Receiving a notification should place the ring into bright mode for certain period of time (15 sec). Colored LED on the surface of the MUUD ring will change its color and stays on for 15 sec with the color specified in the App. The MUUD ring features a colored LED that dynamically changes its color and remains illuminated for 15 seconds when it receives a notification, as specified through the accompanying.

### Mood Ring Color Display Method

According to an embodiment, the processing circuitry may be configured to perform a real time responsive server method including one or more of the following operations: controlling a wearable ring display to display a first color corresponding to a first mood at a first point in time; and controlling the wearable ring display to display a second color corresponding to a second mood at a second point in time, wherein the first color is different than the second color.

FIG. 3A illustrates a flow chart of a real time responsive server method 300 performed by processing circuitry, according to certain embodiments. With reference to FIG. 3A, according to an embodiment, the processing circuitry may be configured to perform a real time responsive server method 300 of controlling a display of a mood ring, including one or more of the following Operations 301-308. For example, the operations may include: collecting (e.g., obtaining/accessing) and storing, by the server, in a database of a memory, ring data (or emotion data) related to a wearable device user's feelings or mood (Operation 301). The wearable device user may be a registered wearable ring device user. The method 300 may further include the server accessing and analyzing the stored ring data (or emotion data) (Operation 302). Based on the analysis, the server may determine a current mood of the wearable device user based on the analyzing of the ring data (or emotion data) (Operation 304).

Operation 304 may include determining what mood score range or category, from a plurality of mood score ranges or categories, the current mood corresponds to, and identifying, as the first color, a color associated with the determined current mood score range or category in the database. The mood may correspond to an overall mood, or be a category specific mood (e.g., fatigue level, mental health).

When the determined current mood corresponds to a first criterion (or mood category), the server may display a first color, such as by controlling the communication interface to transmit information (e.g., graphical user interface (GUI) control information) to a wearable electronic ring device that causes the wearable device to display a first color (Operation 306).

When the determined current mood corresponds to a second criterion (or second mood category), display a second color that is different from the first color (Operation 308). Displaying of the colors may correspond to the responsive server controlling the ring device and/or user terminals to display the respective color.

Operations 304 in conjunction with Operation 306 or Operation 308 may correspond to changing a ring display color according to variations in user activity (detected by the ring device) suggesting a shift in emotional state. Changes in users emotional state are determined by the server based on an interpretation of active and passive ring data.

According to an embodiment, Operation 301 may include one or more of: passive data collection (e.g., collecting sensor data by a sensor(s) of a ring device) and active data collection (e.g., actively collecting information regarding user interaction or activity associated with the wearable device). The collected ring data, which may include sensor data, may be stored in a database of a memory (e.g., of a smartphone or server).

### Passive Data Collection

The mobile ring device may also record biometric data including performing passive data collection, which is data obtained using a variety of electronic sensors located on the ring's inner surface that measure biometric(s) including sensor data that may be used to measure one or more of: heart rate, body temperature, blood oxygen rate and/or respiratory rate. The ring device feeds (transmits) recorded health/sensor information back to the responsive server (and/or smartphone) using software that also modifies the color displayed on the ring's display (e.g., its LED surface). The display color changes according to variations in user mood, which may be based on user activity detected by the ring device suggesting a shift in emotional state. Changes in user's emotional state may be determined by interpretation of one or more of active data and passive data (which each may correspond to user activity). Active data and passive data are explained below, and the data may be combined and weighted for determining an overall mood score.

App-Guided color change may be a primary visual of the ring and a guidance tool for the positive reinforcement system. Color change may occur as either flashes (up to 3), pulses (up to 3) or standing colors-usually depicting formal mood status updates.

Having 6 color intervals on the ring for “Journey” as a marker for progress (1. Plan journey, 2. Start the journey, 3. Finish the journey, 4. Caption the journey, 5. Immediate feedback (MUUD input) and 6. Keywords extra credit: Journaling: “Actual journal entry”).

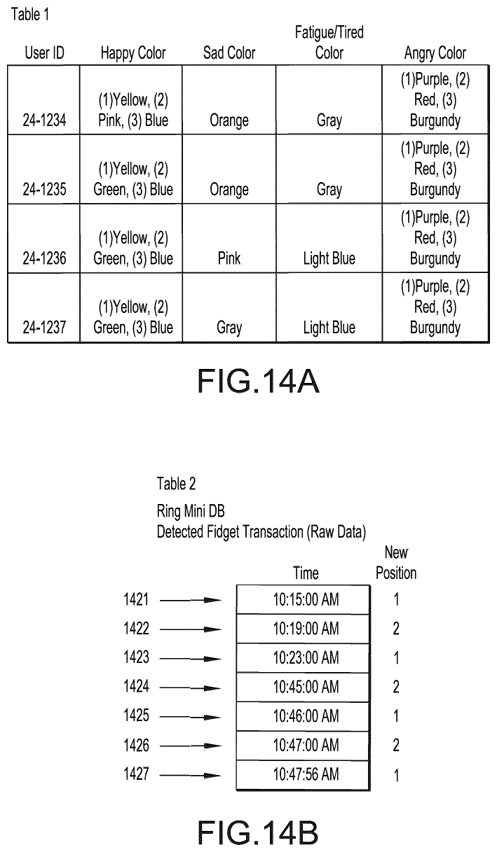

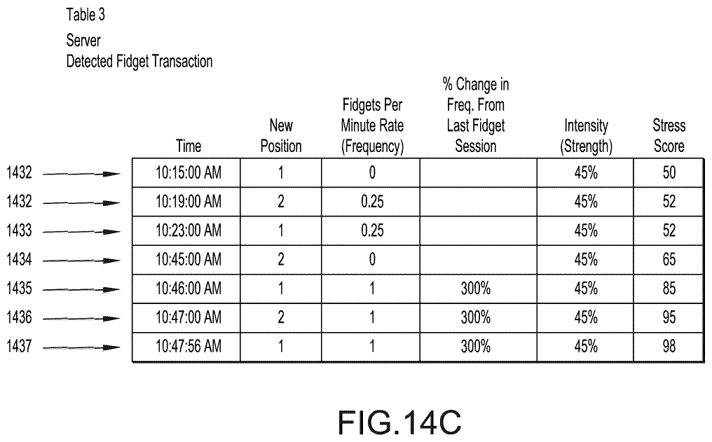

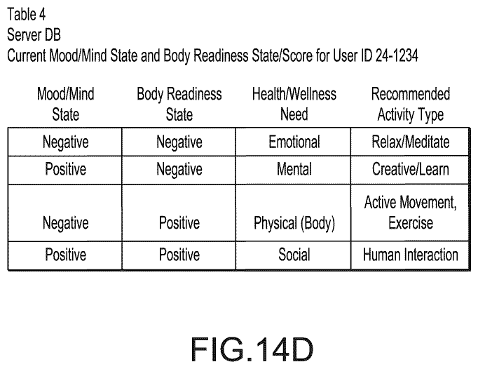

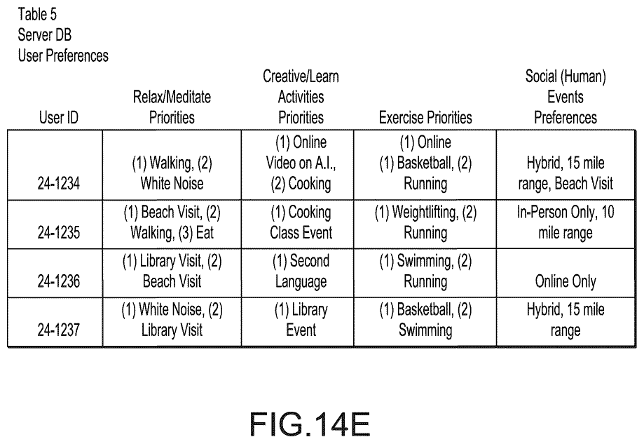

The operations performed by the processor or circuit of the ring device in conjunction with the real time responsive server may include collecting sensor data by the ring device, recording the collected sensor data in a memory, analyzing, or monitoring the recorded sensor data and performing an action in response to a detected condition(s) related to the analyzed/monitored sensor data. The sensor data may be fed back to the responsive server as biometric and/or behavioral health data that can be used to detect increased stress levels to facilitate more timely interventions. The increased stress detection may be associated with the timely interventions. For example, an intervention may mean a recommended “relax/meditate” activity of FIG. 14E. The ring device may be designed to transfer data between software applications linked to smartphones and other related mobile communication platforms that enables recorded data to be downloaded from device to app and uploaded from app to device.

Active Data Collection from Ring: Fidget Activity

Unlike the related art, the ring device's active data collection features represent an approach to behavioral health monitoring and mood tracking by, for example, interpreting changes in frequency and intensity of physical activity over time, which can provide meaningful information about a user's daily stress levels. Anxiety is defined as a feeling of intense, excessive, and persistent worry. Anxiety includes both physical and psychological symptoms precipitated by activation of our fight or flight response (sympathetic nervous system). Physical anxiety symptoms correlate with measurable changes in sensorimotor and muscle activity throughout the body. Examples of motor activity that increase during fight or flight include elevated heart rate, increased ventilation and breathing rates, tremulousness and shivering and restless movements like passive hand fidgeting like finger tapping and variations in the frequency and intensity of these activities are important indicators of stress.

According to an embodiment, Operation 302 includes accessing and analyzing the stored emotion and/or ring data, which may include a process of detecting, based on the ring data, a user's fidget activity over a period of time, and determining a stress level of the wearable ring user based on a frequency of the user's fidget activity over the period of time. The stress level may be used as a factor in determining overall mood and/or readiness.

With reference to FIG. 3B, according to an embodiment, the processing circuitry may be configured to perform a real time responsive server method 350 including one or more of the following Operations 351-358. For example, the operations may include: receiving, from a wearable ring device, a packet including raw digit activity (RDA) (Operation 351); identifying, based on the packet, a registered wearable device user (RWDU) associated with a wearable ring device that transmitted the packet (Operation 352); storing, in a database, the RDA in association with the RWDU (Operation 354); detecting, based on the stored RDA, one or more indications of stress (Operation 356); and determining, based on the one or more indications of stress, an emotional state of the RWDU (Operation 358).

The operations 351-352 may be performed to identify a current mood of the RWDU, and the current mood of the RWDU may be used to perform the color display/change Operations 306/308 based on the current mood. The method 350 may be referred to as active data collection of emotion related data from a wearable ring device.

According to an embodiment, the method 350 may include: (i) identifying a change of a frequency or an intensity of digit activity over time that is above a predetermined frequency or intensity threshold: (ii) identify, based on the identified change of the frequency or the intensity of the digit activity over time being above the predetermined frequency or intensity threshold, an indication of stress; (iii) store the indication of stress in the database in association with the registered RWDU; and (iv) when a number of indications of stress is equal to or greater than a stress threshold, perform an action related to notifying and/or providing a recommendation related to the stress. According to an embodiment, changes in frequency and intensity of physical activity over time may be interpreted to identify and collect more meaningful information about user's daily stress levels. The stress levels may be used to adjust or correspond to a mood score/level.

Active Data Collection from Ring: User Predictive Input

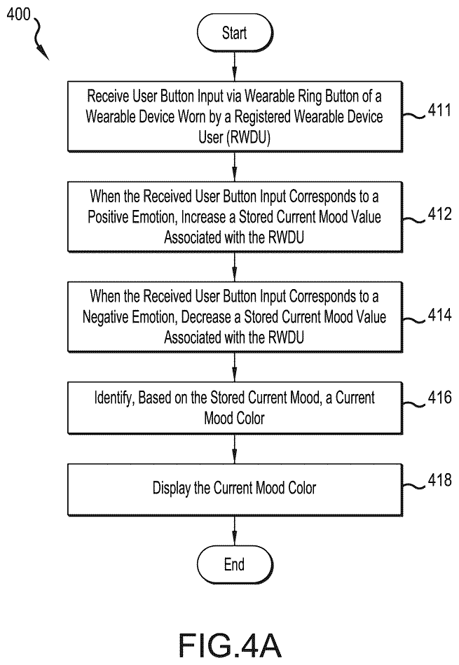

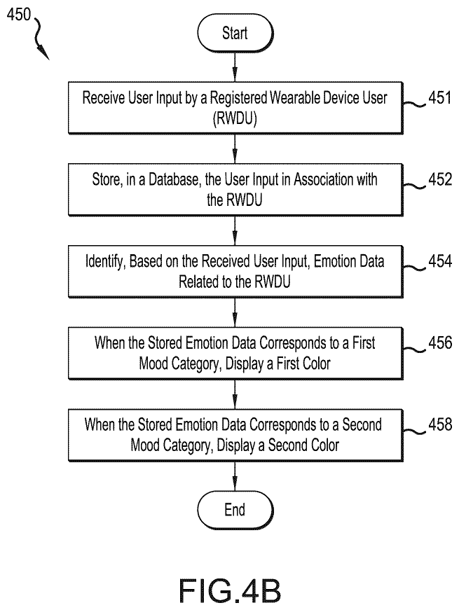

FIG. 4A illustrates a flow chart of a real time responsive server method 400 performed by processing circuitry, according to certain embodiments. The method 400 may include active data collection including collecting emotion-related user input data from a wearable ring device.

With reference to FIG. 4A, according to an embodiment, the processing circuitry may be configured to perform a real time responsive server method 400 including one or more of the following Operations 411-418. For example, the operations of method 400 may include: a user inputting a user input to a wearable ring device via a button (e.g., hard key, touch sensitive panel) provided on the wearable ring device, which causes the button to transmit the user input to the server which receives the user input (Operation 411). Operation 411 may require the user be a registered wearable device user having registration information stored in the database and a validation/log in may be required.

Based on the received user input, when the received user button input corresponds to a positive emotion, the server may increase a stored current mood value associated with the RWDU and store the resulting value as the current mood (Operation 412). In contrast, when the received user button input corresponds to a negative emotion, the server may decrease a stored current mood value associated with the RWDU and store the resulting value as the current mood (Operation 414). Based on the stored current mood, the server may identify a current mood color (Operation 416). The server may cause the current mood color to be displayed by the ring device (Operation 418).

A ring wearer may confirm a positive/negative experience. A main button of a ring may receive a yes/no response as well as positive versus negative experiences confirmed with a swipe up versus down (binary functions).

Separate Casing may be provided. The separate casing includes a separate casing which has a screen and holds the ring for charging, battery charging, helps in increasing the length of time for battery life, storage capacity for data (e.g., 100 GB), safe storage (Very secure and cannot be compromised), ring case can be used to transfer data to someone else's case which elevates the utility of the case (Virtual Diary). Screen for showing emotional expression could be on the casing instead of the ring itself. Thickness by far is the most obstruction and by having a separate case, we can decrease the thickness even further by using smaller size battery in the Ring. Could display MUUD, emojis, friends MUUD, —indicate notifications.

Behavioral enforcement with integrated color change. Basic function of the app (facilitates wellness activity). MUUD. Ring is a conduit to the app by communicating directly with the app to convey progress and creating a reward system for the user to encourage him/her to initiate “journal/journey”.

Fidget tool ties to “Journey” section of MUUD by rotating the ring and each rotation completes each interval of the color change in a journey (6 interval). These 6 intervals have been mentioned in the color change section.

The fidget tool can be used to scroll through more than two items/objects. More intentionality (bilateral-both handed function). More thoughtful. It can be used to rewind the mentioned steps.

Additional features: Data transfer with QR code. Patients can easily transfer their data to doctors using QR code technology by simply scanning their rings.

For journaling, the objective is to make it easier for a user to enter a mood using a predictive text and/or “one button” or “two button” input by allowing emotion input to be formed by a limited number of keypresses. When providing a mood input for a journal, performing a swipe up action on a button changes the wearable ring display's color to a happy mood color (which may be designated as the wearable ring user's happy color, e.g., via one of the Tables 1-5) and the wearable ring user may provide an extent of happiness. That is, when providing a rating/review for journeys, tapping a button a number of times indicates to what extent a user liked or dislike their experience (e.g., the user taps one, two or three times to convey if they feel good, great or the best, respectively, about the experience or encounter they're journaling about). Conversely, swiping down on the button causes a first negative emotion color to be displayed, which can be further scrolled through by tapping. After swiping down, tapping will change the ring color automatically to each of the various pre-programmed colors the user chose for their negative emotion word. Once a mood color has been found, pressing the button, and holding (e.g., for one second) causes the displayed mood color to be selected/entered, and the selection result transmitted to the server. Press and holds for 3 sec turns the device on/off. Press-and holding the primary action button 3 times in a row (one sec holds) generates a non-urgent but important user alert (goes to their inner circle). Pressing and holding the primary action button 3 times in a row (3-sec holds) generates an urgent alert to the user's inner circle.

FIG. 4B illustrates a flow chart of a real time responsive server method 450 performed by processing circuitry, according to certain embodiments. FIG. 4B illustrates a method 450 including: receiving user input from a registered wearable device user (RWDU) (Operation 451); storing, in a database, the user input (or emotion data corresponding to the user input) in association with the RWDU (Operation 452); and identifying, based on the received user input, emotion data related to the RWDU (Operation 454). The method 450 may also include one or more of: when the stored emotion data corresponds to a first mood category or level, display a first color (Operation 456) and when the stored emotion data corresponds to a second mood category, display a second color different from the first color.

The receiving of the user input may correspond to a journal input, which may be received from the ring device (e.g., via a primary or secondary ring button) and/or via a GUI app. The ring device may have a similar and/or complimentary communication structure as the user terminal and/or server for communicating with the server (or user terminals).

The ring device, user terminal and/or server may be configured to: responsive to receiving a user ring input via the wearable ring button, control the network communication interface to transmit the user ring input to a responsive server; receive a response to the transmitted user ring input from the server via the network communication interface; and responsive to receiving the response to the transmitted user ring input from the server, controlling the wearable ring display to display the first color corresponding to the first mood or the second color corresponding to the second mood based on the received response to the transmitted user ring input.

With reference to FIGS. 3A-4B, the first mood category may be different than the second mood category and the first color may be different than the second color. Displaying of the colors may correspond to the responsive server controlling the wearable/ring device and/or user terminals to display the respective color.

Active Data Collection: Collecting Emotion Data from a Smartphone App

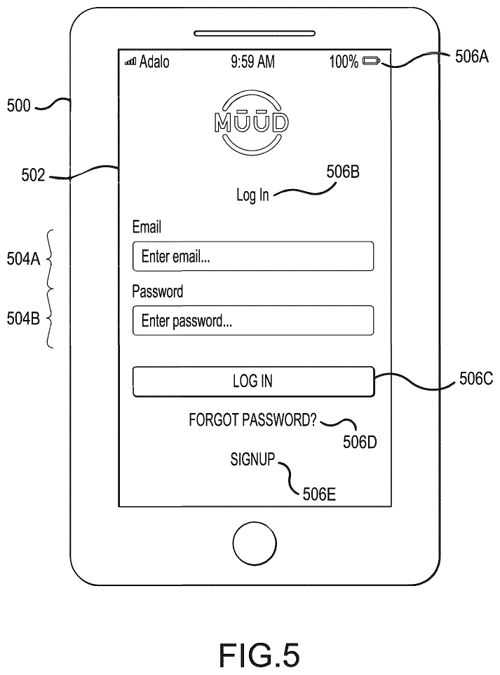

An app/website may also be provided for accessing the database to view and/or augment/update the user's emotion data. For example, FIG. 5 shows a login screen for logging into an app/website, which may be a mood ring related app/website.

FIG. 5 illustrates an electronic display device 500 displaying an example of a mood ring application login GUI 502 (or “login screen”) on an electronic display screen, according to an embodiment. For example, the GUI 502 may also include a first editable user input field 504A for receiving a username and/or email address, and a second editable user input field 504B for receiving a user-entered password. When the login button 506C is selected/clicked, the username/email and password may be transmitted to the server, where the server determines whether the username/email and password correspond to a registered wearable device user (authentication). When the username/email and password correspond to a registered wearable device user, server may determine that authentication succeeded and the user may be logged in, and the home screen (e.g., mood ring home GUI 610 or GUI 620) may be displayed. Touch menu buttons of the login GUI 502 may include one or more of: a log in button 506C, a forgotten password button 506D and a new user signup button 506E. The GUI display 502 may include a status bar 506A, which may be local to the device information, and a screen name (“Log In”). Although selected/clicked are used, an embodiment may require any user interaction such as a gesture. When a hard key button or virtual GUI button is interacted with (e.g., clicked/tapped/touched), a GUI may be displayed. Touched is an example of the button being selected/clicked/interacted with but other user interactions (e.g., tapping, selecting, long pressing, gesture input, voice input, hard key input) may be used to correspond to interacting with, according to various embodiments. Button press/GUI transitions are explained herein. A button's name and/or description may correspond to what GUI transition occurs. For example, when an input or journal input button is pressed, a journal input GUI may be displayed.

After the login button 506C is touched/selected, and the login credentials are successfully validated, a GUI 610 (or “home screen”) may be displayed on the electronic display screen.

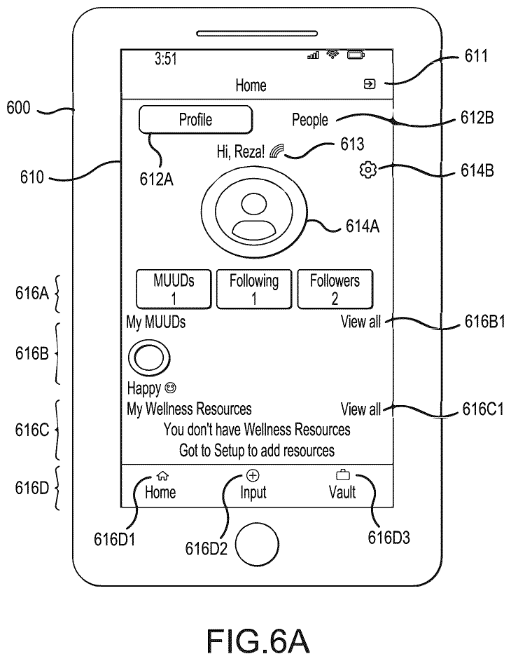

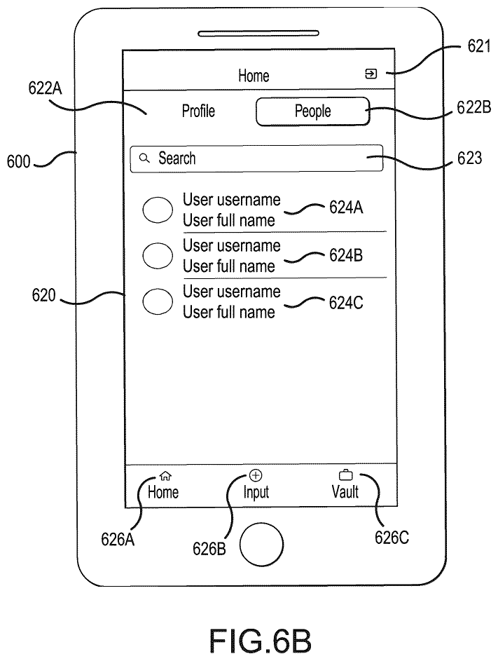

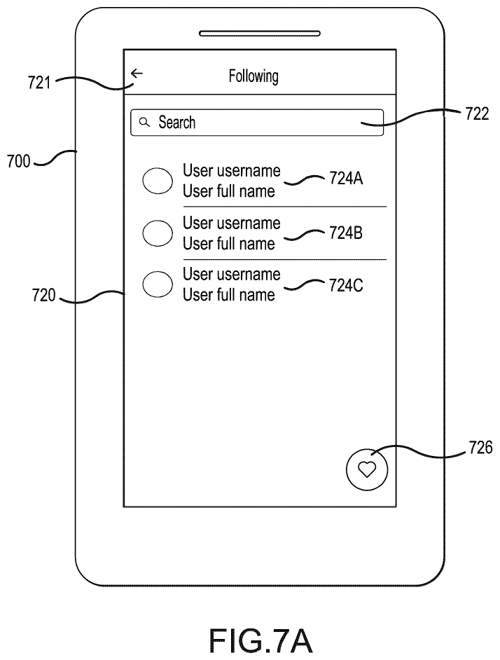

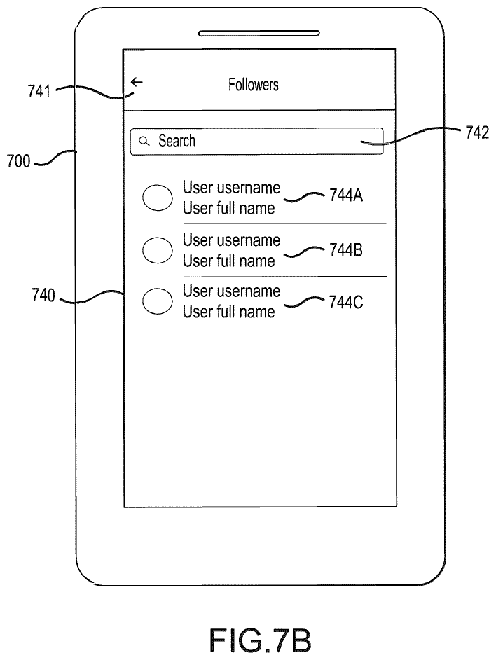

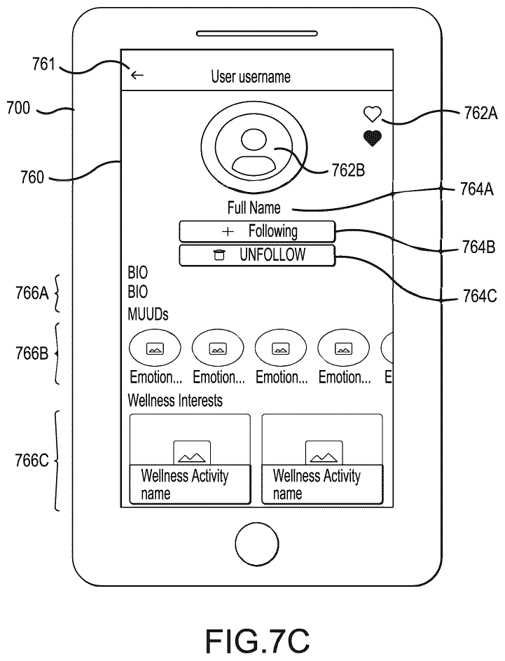

FIG. 6A illustrates an electronic display device 600 displaying an example of a mood ring home GUI 610 (or “home screen”) on an electronic display screen, according to an embodiment. For example, the touch buttons of the home GUI 610 may include one or more of: an exit/return button 611, two toggle buttons (profile toggle button 612A and people toggle button 612B) for toggling between a profile mode and a people mode. In the profile mode (as shown in FIG. 6A), the GUI may display a personalized greeting 613, a profile photo 614A (if available) and a settings button 614B (shown as a gear icon). The settings button 614B may provide an edit screen for modifying the user's profile (such as in GUI 760 of FIG. 7C).

The GUI 610 may also display a dashboard 616A, a registered wearable ring inventory 616B, a wellness resources display area 616C and a footer navigation area 616D.

The dashboard 616A may show three buttons, including wearable device button 616A1 (showing number (i.e., “1”) of registered wearable ring/“MUUDS” devices), a “Following” button 616A2 (showing number (i.e., “1”) of registered users following), and a “Followers” button 616A3 (showing number (i.e., “2”) of followers. The followers and following may be in relation to a social network or social graph.

Button 616B1 and 616C1 are expanded or “view all” buttons. Expand button 616B1 triggers a transition to a GUI that shows all the registered wearable devices. Expand button 616C1 triggers a transition that displays all wellness resources.

The footer navigation area 616D may include a home button 616D1, an input button 616D2 and a vault button 616D3.

FIG. 6B illustrates the home screen GUI 620 in the “people” mode. A difference between FIGS. 6A and 6B is “Profile” mode is toggled on in FIG. 6A and “People” mode is toggled on in FIG. 6B (and the coloring of the buttons 622A, 622B may show which mode is toggled on). The home screen GUI 620 may be displayed in response to the “people mode” GUI button 612B being selected/clicked while GUI 610 is displayed. The “people mode” GUI 620 may display an exit/return button 621, two toggle buttons (profile toggle button 622A and people toggle button 622B) for toggling between a profile mode GUI (FIG. 6A) and a people mode GUI (FIG. 6B). In the people mode GUI (as shown in FIG. 6B), the GUI may display a search box 623 and various usernames 624A, 624B, 624C, which may be search results. The GUI 620 may also include a footer navigation area comprising a home button 626A, an input button 626B and a vault button 626C. The home button 626A may cause a home GUI (e.g., GUI 610 or GUI 620) to be displayed in a mode (either always default the profile mode, or default to a most recent home screen mode).

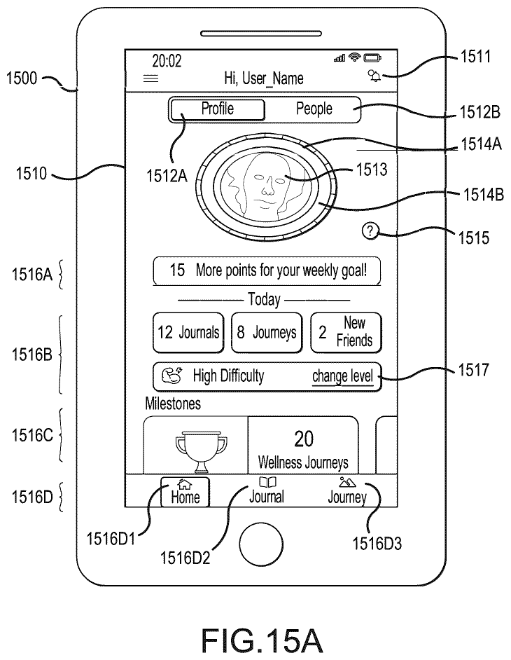

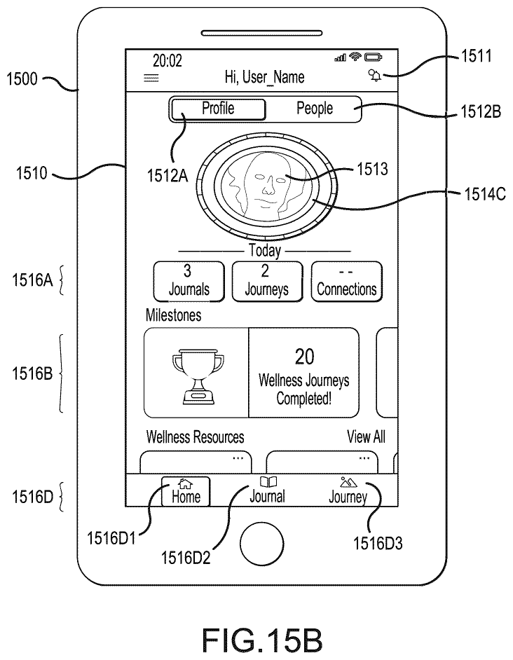

FIGS. 15A and 15B show different versions of profile page GUIs.

FIG. 15A illustrates an electronic display device 1500 displaying an example of a mood ring home GUI 1510 (or “home screen”) on an electronic display screen, according to an embodiment. For example, the touch buttons of the home GUI 1510 may include one or more of: a notifications button 1511, two toggle buttons (profile toggle button 1512A and people toggle button 1512B) for toggling between a profile mode and a people mode. In the profile mode (as shown in FIG. 15A), the GUI may display a profile photo 1513 and an info or help button 1515 (shown as a question mark). The notification button 1511 may provide another GUI or pop-up GUI displaying alerts or notifications, such as when a milestone is completed and/or when a journey is completed. There may be an indicator (e.g., red dot) overlaying a notification button 1511, which may be a bell icon. The red dot may be displayed based on a trigger condition, such as the completion of an objective or task. For example, an objective or task may be a journey, milestone and/or journal entry is completed and/or a predetermined number of a journey, milestone and/or journal entries are completed.

Regarding FIGS. 15A and 15B, the ring device and/or server may be programmed to track a wearer's progress through a journey. In FIG. 15A, profile photo 1513 is surrounded by a green ring 1514B which is surrounded by another outer ring 1514A. The outer ring 1514A may include partially colored in (e.g., shown as purple) broken lines.

The outer ring 1514A includes broken lines depicting the wearer in an active coaching mode indicating that the app is tracking and facilitating their wellness activity performance. When in active coaching mode, the ring device itself may also display the registered ring wearer's journey progress. The partially colored purple broken lines on the app screen provide a virtual representation of what may be depicted on the rings display. The broken lines may be filled in as the user progresses through their journey. The journey is complete when the progress meter comes full circle (i.e., when all potential broken lines are displayed as an indication of all portions are complete). The green ring 1514B in FIG. 15A (the new mock-ups) shows a ring wearer's mood. FIG. 15B shows the ring “full circle”.

The GUI 1510 may also display a journey status display area 1516A (e.g., “15 more points for your weekly goal!”) and a dashboard display area 1516B. The dashboard display area 1516B may include statistics related to the ring wearer's journey. For example, dashboard display area 1516B may include a number of journal entries (e.g., “12”), which may be a cumulative total and/or a total of entries for the current journey. The dashboard display area 1516B may also include a number of journeys (e.g., “8”) and a number of new friends (e.g., “2”). The GUI 1510 may also display difficulty level 1517 that displays the current difficulty level (e.g., “High Difficulty”) of the current journey and/or a change level button that can be clicked/tapped to change the difficulty to level to a different level (e.g., Easy or Low Difficulty).

Milestones display area 1516C shows milestones such as an award(s) or badge(s), such as “20 Wellness Journeys” adjacent to a gold trophy, which means 20 wellness journeys have been completed. The footer navigation area 1516D may include a home button 1516D1, a journal input button 1516D2 and a journey button 1516D3. The buttons of the footer navigation area 1516D may perform the same and/or similar functions as the buttons of other footer navigation areas.

FIG. 15B illustrates an electronic display device 1500 displaying an example of a mood ring home GUI 1510 (or “home screen”) on an electronic display screen, according to an embodiment. For example, the touch buttons of the home GUI 1510 may include one or more of: a notifications button 1511, two toggle buttons (profile toggle button 1512A and people toggle button 1512B) for toggling between a profile mode and a people mode. In the profile mode (as shown in FIG. 15B), the GUI may display a profile photo 1513. The notification button 1511 may provide another GUI or pop-up GUI displaying alerts or notifications, such as when a milestone is completed and/or when a journey is completed.

In FIG. 15B, solid purple ring 1514C shows an indication/alert/notification of a user's mood and/or that a journey, journey, or milestone has been completed (e.g., recently completed; recently may mean within a predetermined amount of time such as over last 4 hours, or last 24 hours, etc.). Solid purple ring 1514C may be displayed when a condition is met, such as a journey, journal or milestone is complete. The solid purple ring 1514C may show a progress meter at full circle.

The GUI 1510 may also display a journey status display area 1516A (e.g., “15 more points for your weekly goal!”) and a dashboard display area 1516B. The dashboard display area 1516B may include statistics related to the ring wearer's journey. For example, dashboard display area 1516B may include a number of journal entries (e.g., “12”), which may be a cumulative total and/or a total of entries for the current journey. The dashboard display area 1516B may also include a number of journeys (e.g., “8”) and a number of new friends (e.g., “2”). The GUI 1510 may also display difficulty level 1517 that displays the current difficulty level (e.g., “High Difficulty”) of the current journey and/or a change level button that can be clicked/tapped to change the difficulty to level to a different level (e.g., Easy or Low Difficulty).

Milestones display area 1516C shows milestones such as an award(s) or badge(s), such as “20 Wellness Journeys” adjacent to a gold trophy, which means 20 wellness journeys have been completed. The footer navigation area 1516D may include a home button 1516D1, a journal input button 1516D2 and a journey button 1516D3. The buttons of the footer navigation area 1516D may perform the same and/or similar functions as the buttons of other footer navigation areas.

FIG. 1B illustrates the home screen GUI 620 in the “people” mode. A difference between FIGS. 6A and 6B is “Profile” mode is toggled on in FIG. 6A and “People” mode is toggled on in FIG. 6B (and the coloring of the buttons 622A, 622B may show which mode is toggled on). The home screen GUI 620 may be displayed in response to the “people mode” GUI button 612B being selected/clicked while GUI 610 is displayed. The “people mode” GUI 620 may display an exit/return button 621, two toggle buttons (profile toggle button 622A and people toggle button 622B) for toggling between a profile mode GUI (FIG. 6A) and a people mode GUI (FIG. 6B). In the people mode GUI (as shown in FIG. 6B), the GUI may display a search box 623 and various usernames 624A, 624B, 624C, which may be search results. The GUI 620 may also include a footer navigation area comprising a home button 626A, an input button 626B and a vault button 626C. The home button 626A may cause a home GUI (e.g., GUI 610 or GUI 620) to be displayed in a mode (either always default the profile mode, or default to a most recent home screen mode).

FIGS. 7A and 7B illustrate a categorized search interface, according to an embodiment. GUI 720 of FIG. 7A shows a list of users 724A, 724B, 724C that the logged in user is following, and GUI 740 of FIG. 7B shows a list of users 744A, 744B, 744C following the logged in user. FIGS. 7A and 7B may include some or all of the functionality as in the GUI of FIG. 6B. For example, the search input fields may be configured to perform a search in relation to (or limited by) the type of GUI being displayed. That is the following GUI 720 may limit any searches to followed/following connections only, and the follower GUI 740 may limit any searches to followers only.

FIG. 7C illustrates a GUI 760 including an exit/back button 761, a like indicator 762A (which may be hearts), a profile picture 762B, a full name 764A, a follow button 764B, an unfollow button 764C, a bio description area 766A, emotional data entry (or MUUD entries) area 766B, and a wellness activity area 766C. When the button 764B is clicked, the named 764A connection is added to the RWDU following connection list/social graph. When the button 764C is clicked, the named 764A connection is removed from the following connection list/social graph.

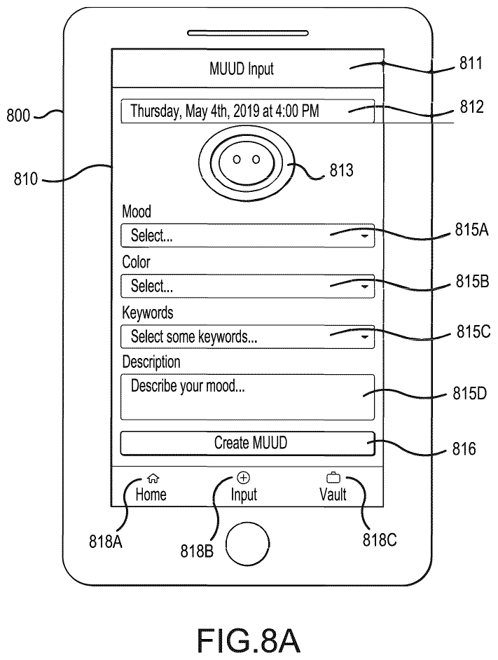

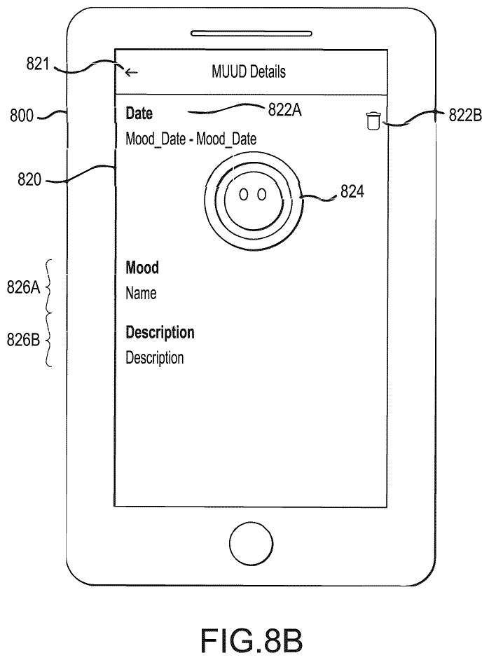

FIG. 8A illustrates an input screen 810 for entering a mood input, according to an embodiment. When an input button is clicked/tapped, the input screen 810 may be displayed. For example, an electronic display device 800 may display a MUUD input GUI 810 that includes a title 811, a date/time 812 (which may be a current date/time or user input date/time), a mood indicator face 813, a mood drop-down list 815A, a color drop-down list 815B, a keyword selection drop-down list 815C, and a description text entry box 815D. The GUI 810 may also include a “create” button 816 for saving/creating the MUUD/mood input entry. Buttons 818A-818C may correspond to buttons 626A-626C, respectively.

FIG. 8B illustrates a GUI 820 that shows a finalized individualized view of a MUUD entry detail screen. For example, GUI 820 shows a back button 821, a date/time area 822A, a delete button 822B, a visual representation of a mood status 824, a mood name 826A and a description 826B. The information entered into the GUI of FIG. 8A may be displayed in FIG. 8B in response to the create button 816 being clicked/pressed/touched.

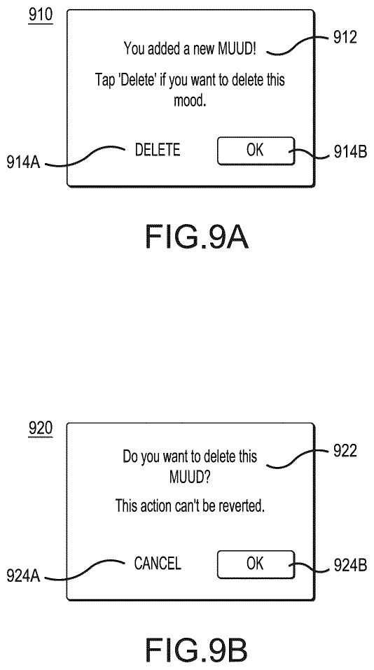

FIGS. 9A and 9B illustrate pop-up confirmation screens that may be displayed overlaying one or more GUI screens when an action is or has been performed. For example, pop-up screen 910 displays a text 912 (e.g., “You added a new MUUD! Tap ‘Delete’ if you want to delete this mood.”) and a delete button 914A and an OK button 914B. Pressing a delete button deletes the new entry and OK stores the new entry in the database.

GUI 920 illustrates a delete confirmation pop-up which may be displayed when a delete button is tapped/selected. For example, the pop-up screen 920 may display text (e.g., “Do you want to delete this MUUD? This action can't be reverted.”) and a cancel button 924A and an OK button 924B. GUI 920 may be displayed when a delete, cancel or back button is clicked/tapped/touched.

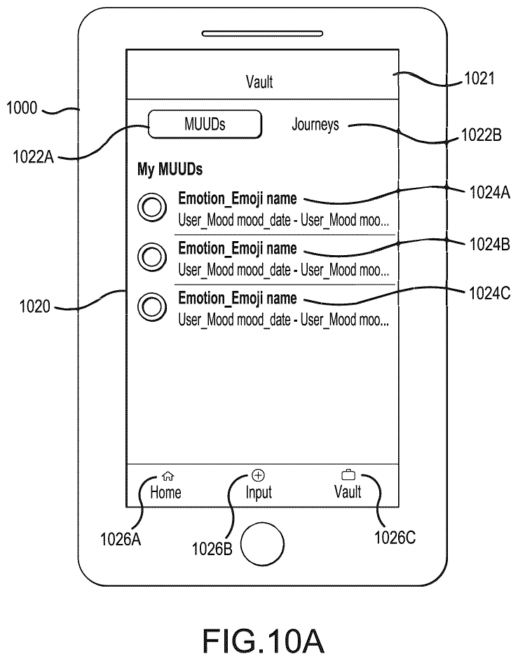

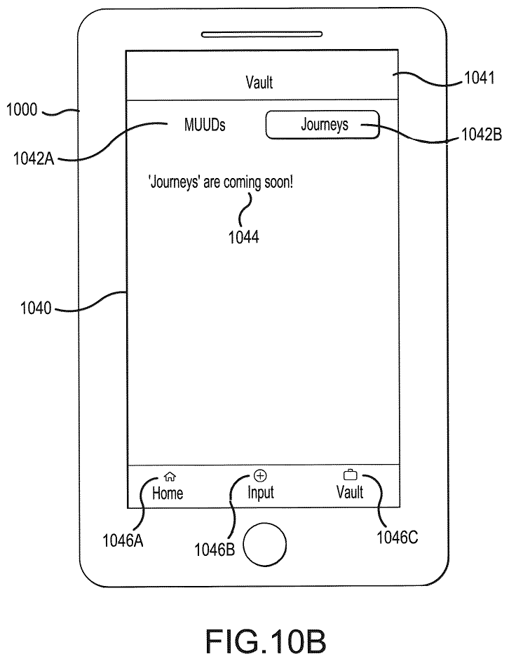

FIG. 10A illustrates a mood-related input history screen 1020, which may be referred to as a first vault. FIG. 10B illustrates a journey input history screen 1040, which may be referred to as a second vault. The transition of toggling between a mood-related screen (FIG. 10A) and a Journeys screen (FIG. 10B) is performed via the buttons 1022A/1022B and 1042A/1042B.

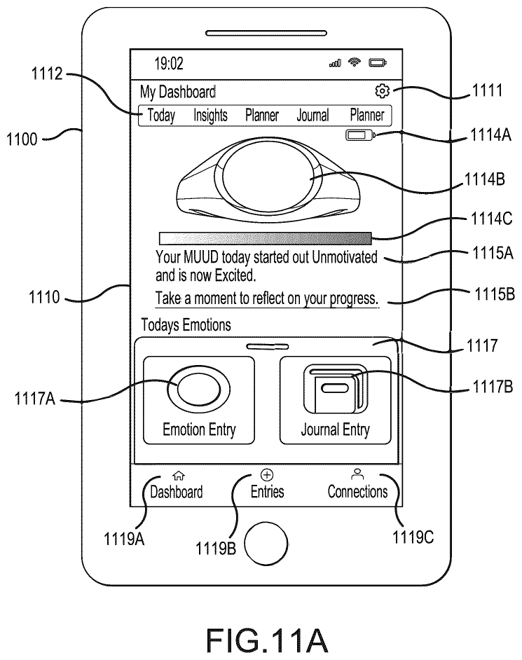

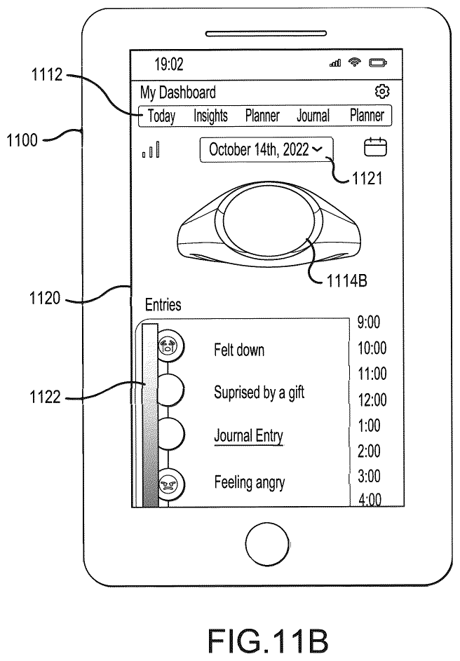

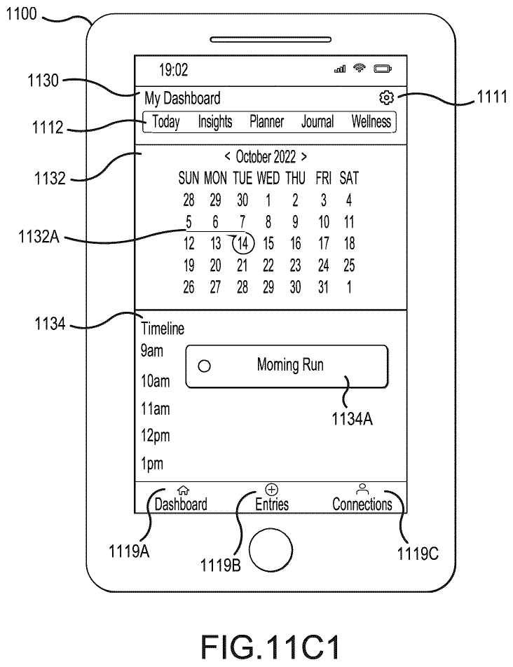

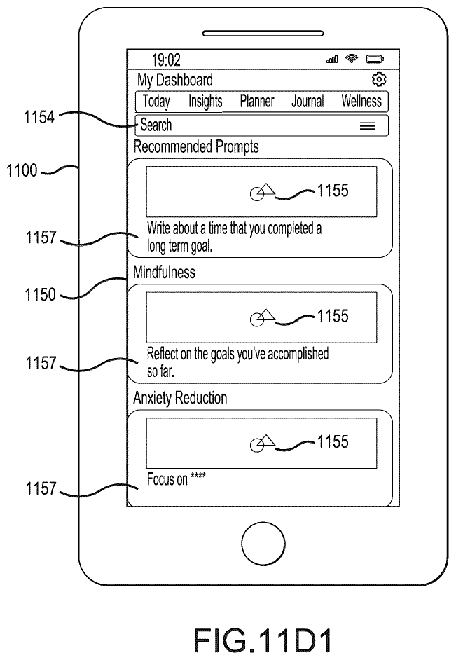

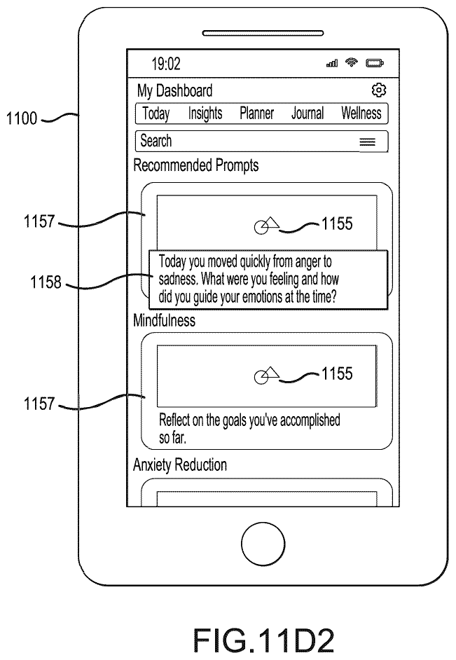

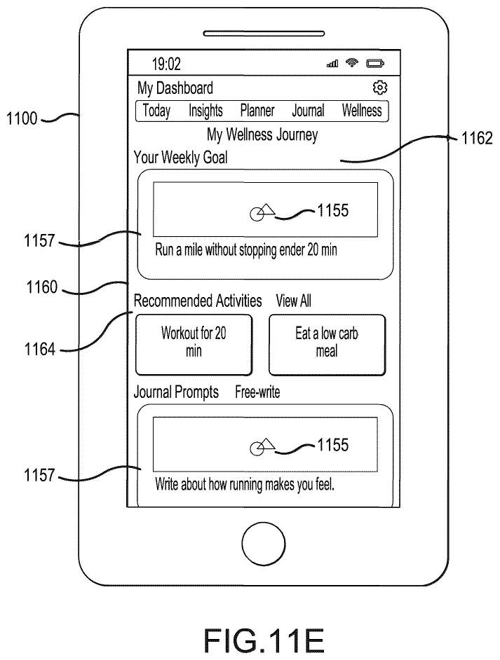

GUI 1020 illustrates a list of visual representations 1024A, 1024B, 1024C of emotion (e.g., mood color and corresponding emoji). The emotions may each correspond to a different wearable device and/or may correspond to different points in time. FIG. 10B illustrates a journeys interface which is further described with reference to FIGS. 11A-11E.

FIGS. 11A-11E illustrate a wearable ring mobile application including a dashboard navigation experience. Each of the GUIs in the dashboard may include a GUI navigation bar 1154 with five tabs. The GUI navigation bar may be provided in an upper area of the GUI.

The five tabs may include text buttons corresponding to each of a “Today summary” GUI 1119 (FIG. 11A), an Insight GUI 1120 (FIG. 11B), a planner GUI 1130 (FIGS. 11C1 and 11C2), a journal GUI 1140 (FIGS. 11D1 and 11D2) and a wellness activities GUI 1150 (FIG. 11E). Clicking or tapping on the identified button causes the screen to transition to the corresponding GUI.

According to an embodiment, FIG. 11A illustrates an example dashboard daily GUI 1110, which may be referred to as a daily summary or “Today summary”. The daily GUI 1110 may include and display a settings gear icon 1111, a dashboard menu bar 1112, a battery icon 1114A, a ring icon 1114B, a color bar 1114C, an insight 1115A, a journal hyperlink 1115B, a today's emotions display area 1117 and an app navigation bar 1119 that is comprised of dashboard button 1119A, entries button 1119B and connections button 1119C.

The battery icon 1114A may indicate the state of charge of a battery (battery level) of a wearable ring display device. As shown in FIG. 11x, the battery icon 1114A may be a second battery icon displayed, as the upper right corner of the display screen may indicate the battery level of the user terminal (e.g., smartphone).

The ring icon 1114B may be a visual representation (e.g., a ring display device icon with changing display colors) and/or or with the currently displayed color of the wearable ring device. The color bar 1114C may correspond to a user's emotion or mood record over the current day.

Insight 1115A may comprise displayed text corresponding to an emotional state of a user. For example, the insight 1115A may state that the user's mood “started out unmotivated and is now excited.” Insight 1115A may include discussion of two moods/emotional states from two different periods of time within a certain time period (e.g., the same calendar day, or the same 24-hour period).

The journal hyperlinks 1115B and 1117B may include text (e.g., “Take a moment to reflect on your progress”, or “Journal Entry”) and, when clicked/tapped transition the application to cause a journal entry input GUI 1140 to be displayed.

Today's emotions display area 1117 may display certain background/theme colors in the app that match a current mood. The display area 1117 may include an emotion entry button 1117A and a journal entry 1117B. The emotion entry button may cause an emotion input and/or journal input GUI to be displayed.

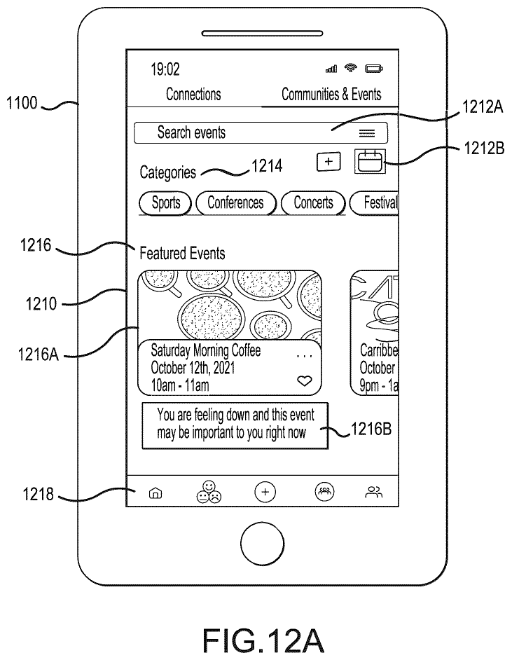

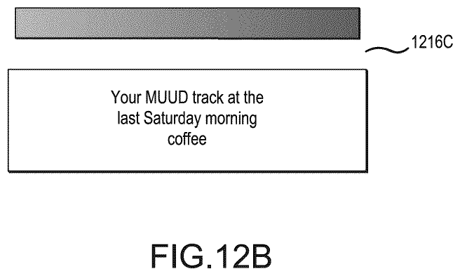

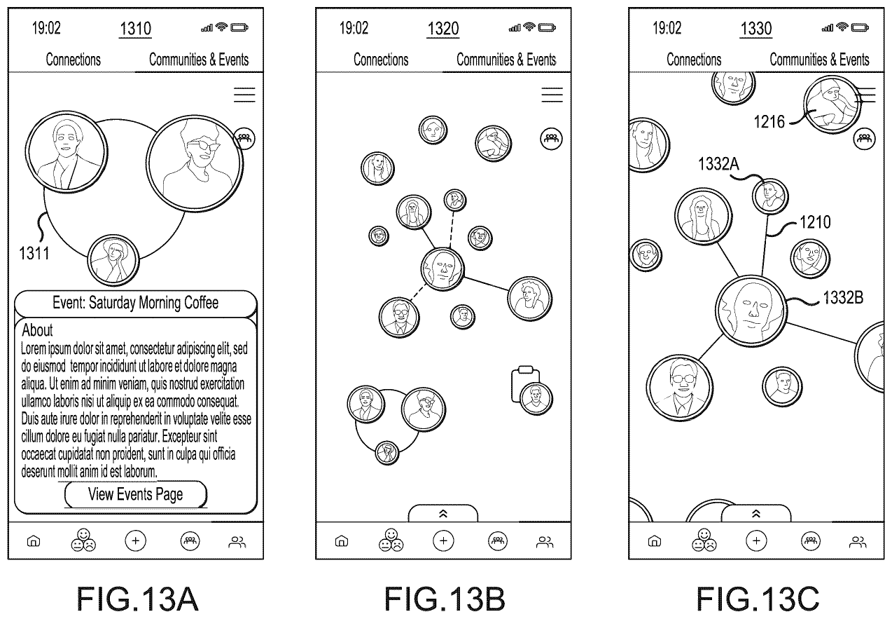

The lower app navigation bar 1119 may be comprised of dashboard button 1119A, an entry button 1119B and a connections button 1119C. The dashboard button 1119A is shown as activated in FIG. 11A. The lower app navigation bar 1119 allows for quick switching between different modes of the app. Entries button 1119B navigates the app to a GUI corresponding to an input screen, and connections button 1119C navigates the app to a connections GUI, such as in FIG. 12A and/or FIG. 13A-13C.

FIG. 11B illustrates an insights GUI 1110, according to an embodiment. The insights GUI 1130 may include the dashboard menu bar 1112 and the ring icon 1114B, as well as a calendar button 1121 and a tracked mood display area 1122, which may be referred to as temporal/timeline insights 1122. The calendar button 1121 may cause a calendar input GUI pop-up to be displayed, such as a calendar entry GUI and a drop-down list of calendar items (e.g., weekly, monthly, and yearly and/or date ranges corresponding to a period of time, such as last week, last month, etc.). The timeline insights 1122 may include emojis (e.g., felt down/sad emoji, angry emoji, happy emoji) and mood-based colors as a way to provide tracking of the registered user's mood/emotions over time. The calendar button 1121 is configured to, based on user input, provide temporal filtering of the data used to provide the timeline/temporal insights 1122 (i.e., the tracking in the display area 1132 is filtered based on date selected by user input using calendar button 1121, which may be a drop-down or calendar menu 1121). This feature allows tracking of mood by time and providing daily, weekly, monthly and/or yearly mood tracking for display. The temporal insights/display area 1122 displays four mood entries, ordered by time, including “felt down” text with emoji and mood color blue, a “surprised by gift” text with mood color purple, a “Journal Entry” text (which may be hyperlinked back to the original entry for viewing) with mood color orange, and a “feeling angry” text with mad face emoji overlaying red circle background.

FIGS. 11C1 and 11C2 illustrate a planner or calendar GUI 1130, according to an embodiment(s). The planner GUI 1130 may include a calendar GUI 1132 including an indication 1132A of a selected day (which may default to a current day before selection, the selection may be persistent or not persistent). The planner GUI 1130 may also include a timeline 1134 corresponding to any scheduled activities/meetings/appointments for the selected day (the schedule may be imported from third-party service providers). For example, a “morning run” may be scheduled for 9:00 AM-10:00 AM on the selected day 1132A (shown as Oct. 14, 2022, in the calendar GUI 1132). FIG. 11C2 shows a pop-up dialogue box 1134 that may provide a mood insight based on a current mood and the scheduled activity. For example, the pop-up dialogue box may include the text: “You're feeling a bit down so this run may feel harder than usual and that's okay!” As shown in the drawings, the timeline(s) may include a background color corresponding to a mood at that time. For example, FIG. 11C2 shows a timeline 1134 having a background color that changes over the timeline. The planner brings some insight into the planned/scheduled activity according to the user's mood.

FIGS. 11D1 and 11D2 illustrate a journal GUI 1140, according to an embodiment(s). The journal GUI 1140 may include a search box 1154, various editable user input boxes 1155 and prompts 1157. The search box 1154 may accept user input for searching through stored journal entries. The editable user input boxes 1155 may capture a user input (e.g., text, image, voice memo), which may be in response to a prompt 1157. For example, a prompt 1157 may recite “Write about a time you completed a long-term goal”. The user may enter user input 1155, which may be stored in association with or without the prompt 1157. The user input 1155 may be used to gauge a user's emotion/mood. FIG. 11D2 shows a pop-up window 1158 that provides an emotion/mood insight. For example, pop-up window 1158 may recite “Today you moved quickly from anger to sadness. What were you feeling and how did you guide your emotions at the time?”

### Predictive or “One Button” User Input

A journal entry may also be entered using the wearable ring device (or via a GUI). Predictive commands may correspond to commands performed with only one button or one touch sensitive panel. When providing a mood input for a journal, the wearable ring device may recognize various touch inputs, such as swipes and taps. Like predictive text technology, the button of the wearable device may provide the ability to communicate using one button. This has the benefit of keeping the ring device compact (by not having a multitude of buttons) while also enhancing the robustness (and hence accuracy) of collected emotion related data. In particular, a detected swipe up (e.g., when a finger is detected swiping up on a button or touch sensitive panel) may cause the display to change colors (e.g., to display a happy color). Swiping up/Swiping down may allow the user to cycle the display through various colors corresponding to various moods.

While displaying the happy color, the wearable ring may receive an indication of intensity level, by detecting taps. For example, one or more taps may be detected, and the number of taps may vary. The number of taps may convey varying intensities of emotions corresponding to the displayed mood color. For example, detecting a single tap (alone) may convey “feel good”, detecting a double tap may convey “feel great” and detecting a triple tap may convey “feel the best.” The single tap may correspond to a tap that is not followed by another tap quickly after the first tap (where quickly corresponds to within a predetermined period of time, which may be about one second). A double tap requires two taps detected within the predetermined short period of time. Two taps outside of the predetermined window would be considered single taps. A triple tap includes three taps within a predetermined second time window, which may be the same or longer than the first-time window.

The captured intensity and mood (corresponding to displayed color) may be entered as user mood and/or journal input by using the “one button” predictive input discussed above. That is, the input data may be associated with the user mood and an intensity of the mood. As discussed above, the wearable ring device may detect a “swipe up”, which may toggle and/or scroll through a predefined and/or pre-selected list of mood colors, which may be a group of colors, such as “happy emotion” colors. The detected tap type (e.g., single tap, double tap, triple tap) may be used to enter/select the toggled/scrolled to mood color. This input may convey information regarding how the user feels about an experience or encounter they are journaling about.

Conversely, based on detecting one or more swipe down inputs, the display color scrolls through a different group of mood/emotion colors, such as the user's negative emotion colors by tapping. After swiping down one or more times, tapping will change the ring color automatically to one of the various pre-programmed colors the user chose for their emotion words. After selection, a press and hold (one sec) input may be detected for selecting and storing the scrolled to emotion color and the associated intensity. That is, press and hold for one second may be a select/enter function. Press and hold for three seconds may turn the ring device on/off (power on/off).

Pressing and holding both action buttons simultaneously for 3 seconds sends out an emergency signal that dials 9-1-1 and sends out a distress signal to inner circle and friends.

The button may include additional functionality including a detected user input of “Press-and holding the primary action button three times in a row, where each hold is one second or approximately one second (e.g., ‘one second holds’)”, may generates a non-urgent but important user alert (e.g., broadcast/goes to the RWDU's inner circle). “Press and holding the primary action button three times in a row for a second longer period of time (e.g., three seconds or approximately three seconds (3-sec holds)), may generate an urgent alert to the user's inner circle (which may correspond to a short list of connections in the social graph).

FIG. 11E illustrates a wellness GUI 1160, according to an embodiment. The wellness GUI includes various activities for improving mental wellness including a weekly goal 1162 and recommended activities 1164. The weekly goal 1162 may be related to exercise, such as “run a mile without stopping under 20 min”. The recommended activities 1164 may be related to exercise and/or nutrition, such as “workout for 20 min” and/or “eat a low carb meal.”

FIG. 12A illustrates a communities & events GUI 1210, which includes a search box 1212A, a calendar button 1212B, a categories display area 1214, a featured events display area 1216. The search box 1212A may cause a search GUI to pop-up, which provides the functionality for a user to input a text search for events. The calendar button 1212B may cause a calendar or planner GUI to be displayed when the button is clicked/tapped. The categories display area 1214 may include event category filters (e.g., Sports, Conferences, Concerts, Festivals), which correspond to selectable elements, which filters the events displayed in the featured events display area 1216. The featured events display area 1216 may include an event thumbnail 1216A, which may be clicked on to see more details about the event. The display area 1216 may also include an event insight 1216, such as “You are feeling down and this event may be important to you right now”, which may be associated with an event displayed in the featured events display area. The insights discussed herein may include displaying text (e.g., in a pop-up window) or an audio speech output of the text insight (e.g., using text to speech technology). FIG. 12B illustrates an alternative type of text insight 1216C that includes a color bar showing a mood over time corresponding to a similar event (or the same recurring event in the past) and text that recites “Your MUUD track at the last Saturday morning coffee” or the like.

FIGS. 13A-13C illustrate a connections user interface/user experience (UI/UX) that includes a zoomed in connections GUI 1310, a zoomed-out GUI 1320, and a regular view 1330. Each of the GUIS 1310-1330 may include color coded moods surrounding various user profile photos. For example, a user having a good mood may have a bright happy color embodied by a yellow ring around their profile photo, while others may have a red color ring around their profile photo when that user is in a bad mood.

The zoomed in connections GUI 1310 may display information regarding a single event, including information about the event, and a hyperlink to an events page. The GUI 1310 may also include an indication 1311 of a relationship (e.g., a circle connecting event attendees). The GUI 1320 may show all connections, and the GUI 1330 may allow for zooming in to see connections closer.

FIGS. 14A-14E illustrate various data structures and/or data tables that may be used in conjunction with the above-described methods and components. For example, FIG. 14A illustrates a Table 1 that may be a color correspondence table and include a user ID column, a happy color column, a sad color column, a fatigue/tired color column and an angry color column. Table 1 may correspond to user-entered preferences that associate one or more colors (e.g., blue) with a mood (e.g., happy) on a personalized/customizable level. That is, a user may adjust which color they associate with which mood. Moreover, this information related to associations may be learned by the system based on user inputs that, for example, explicitly or implicitly associate a certain color with a certain mood/feeling. As shown in Table 1, a user may have more than one color associated with an emotion/mood color. For example, User ID 24-1234 includes Yellow and Pink as happy colors, and red and burgundy as angry colors.

FIG. 14B illustrates Table 2 including detected fidget transaction data (raw data0 that may correspond to detected fidget activity and a timestamp of the detected activity. For example, entries 1421-1427 of Table 3 each include a timestamp during the 10 o'clock hour and include a position of the fidget detector (e.g., a position of a sliding element acting as a fidget detector). FIG. 14C illustrates Table 3, which may be stored in the server's database. Table 3 includes analyzed entries 1431-1437. Entries 1431-1437 may be based on and incorporate the raw data from entries 1421-1427 and also add additional supplemental information. For example, Table 4 shows a fidgets per minute rate (frequency) column, a percent change in frequency from last fidget column, an intensity (strength) column and a stress score column. As shown in Table 3, entries 1435-1437 included an unexpectedly large increase in frequency of fidget activity between 10:46 and 10:48 AM, which may indicate this was a stressful event or time of day for the RWDU. The stress score columns also may reflect a level of stress that is based on the fidget frequency and/or an intensity/strength of the fidget activity.

FIG. 14D illustrates a dual correspondence table (Table 4) that provides mind/body conditions for identifying how to treat a combination mind and body state. For example, Table 4 shows the following conditions: Condition 1 (Negative Mood/Mind State and Negative Body Readiness State condition), Condition 2 (Positive Mood/Mind State and Negative Body Readiness State condition), Condition 3 (Negative Mood/Mind State and a Positive Body Readiness State condition), and Condition 4 (Positive Mood/Mind State and Positive Body Readiness State condition). As shown in Table 4, the conditions may correspond to a health and wellness need or category and an associated recommended activity type for that need or category. For example, Condition 1 may correspond to emotional need category and an associated recommended activity of relax/meditate, Condition 2 may correspond to a mental need category and an associated recommended activity of creative/learn, Condition 3 may correspond to a physical (body) need category and an associated recommended activity of active movement/exercise, and Condition 4 may correspond to a social need category and an associated recommended activity of human interaction need category. The data in Table 4 may be used to process any emotion data. For example, the emotion data may comprise one or more of active data collection data, passive data collection data, and an emotional score of wellness entries and/or wellness journey types.

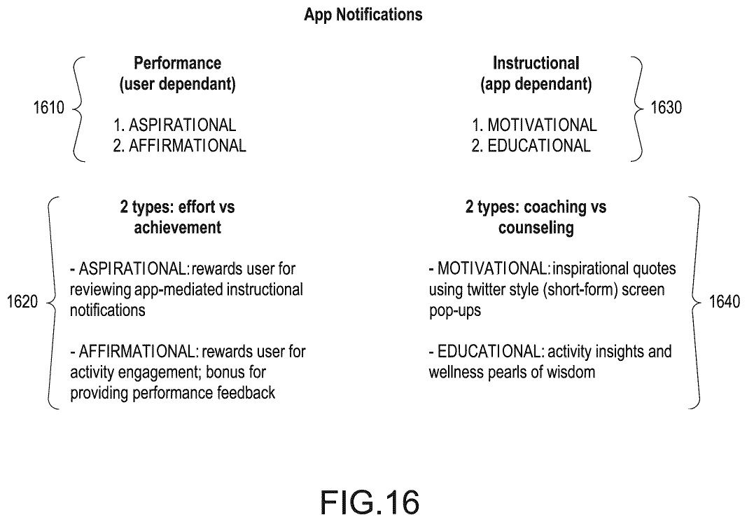

FIG. 16 illustrates App Notifications/Alerts groups, according to an embodiment. For example, a first app notification/alert grouping 1610 may include: Performance (User Dependent) including Aspirational and/or Affirmational. Performance group 1610 may include two types of goals 1620 including Effort and Achievement. For the group of ASPIRATIONAL, a user may be rewarded for reviewing app-mediated instructional notifications, and for AFFIRMATIONAL, a user may be rewarded for activity engagement, including a bonus for providing performance feedback.

A second app notification/alert group 1630 may be Instructional (App Version Dependent): MOTIVATIONAL and EDUCATIONAL. The instructional group 1630 may include two types: coaching, counseling. MOTIVATIONAL: inspirational quotes using twitter style (short form) screen pop-ups; EDUCATIONAL: activity insights and wellness pearls of wisdom.

FIG. 14E shows Table 5 that provides various user IDs associated with the recommended activity types of Table 4. For example, User ID 24-1234 is associated with (1) Walking, and (2) Running in that order for relax/meditate. Thus, when the user corresponding to User ID 24-1234 is detected as being in a negative mood/mind state and a negative body readiness state, the GUI of the User ID may display recommendations related to walking first, playing white noise second, and so on. The other activities in Table 5 show priorities associated with the recommended activity type (and/or the health/wellness need) in a database such that the user's preferences and priorities may be considered to provide a personalized GUI interface.

The Tables 1-5 may be data tables or databases, including logical relationships/associations used for performing the various methods (including user display experiences/navigation) discussed herein. The data tables and/or database(s) may be stored on an electronic device, such as the server(s), user terminal(s) and/or wearable(s).

Advantages. Dedicated to helping people live happier & healthier lives, using technology as a tool for personal wellness. By creating better ways for people to communicate about their activities while connecting with others, the devices and systems aim to inspire unity and understanding that cultivates communities of kindness.

Overlapping elements are not repeated and may correspond to previously described elements having a same or similar name. While explaining the drawings according to embodiments of the disclosure, a detailed description of the contents overlapping with the drawings according to the embodiments is omitted.

Although exemplary embodiments have been shown and described, the disclosure is not limited thereto, and various modifications and variations are possible within the scope of the disclosure. It will be appreciated by those skilled in the art that changes may be made in these exemplary embodiments without departing from the principles and spirit of the general inventive concept, the scope of which is defined in the appended claims and their equivalents.

1. A wearable electronic ring device comprising:
a wearable ring body housing, wherein the body housing has a cylindrical shape and a first outer surface configured to engage with a digit of a dactyl mammal as a ring, and the first outer surface is firmly attachable to the digit of the dactyl mammal as the ring;
a wearable ring display;
a wearable ring memory; and
wearable ring processing circuitry, wherein the wearable ring processing circuitry is configured to execute computer-executable instructions stored in the wearable ring memory to perform the following operations:
controlling the wearable ring display to display a first color corresponding to a first mood or emotion at a first point in time; and
controlling the wearable ring display to display a second color corresponding to a second mood or emotion at a second point in time, wherein the first color is different than the second color, wherein the displayed ring color is associated with a current feeling.

2. The wearable electronic ring device according to claim 1, further comprising a wearable ring network communication interface and a wearable ring button provided on a second outer surface of the wearable ring housing, wherein the computer-executable instructions stored in the wearable ring memory further include instructions configured to cause the wearable ring processing circuitry to perform the following operations:
responsive to receiving a user ring input via the wearable ring button, controlling the network communication interface to transmit the user ring input to a responsive server;
receive, via the network communication interface, a response to the transmitted user ring input from the server; and
responsive to receiving the response to the transmitted user ring input from the server, control the wearable ring display to display the first color corresponding to the first mood or the second color corresponding to the second mood based on the received response to the transmitted user ring input.

3. The wearable electronic ring device according to claim 2, wherein the controlling of the wearable ring display to display the first color corresponding to the first mood or the second color corresponding to the second mood based on the received response to the transmitted user ring input includes controlling the wearable ring display to change a color of the wearable ring display.

4. The wearable electronic ring device according to claim 3, wherein the controlling the wearable ring display to change a color of the wearable ring display includes outputting a notification indicating the color change.

5. The wearable ring device according to claim 4, wherein the outputting of the notification indicating the color change includes at least one of: controlling the wearable ring display to flash, controlling the wearable ring display to pulse, or controlling the wearable ring to output haptic feedback.

6. A real time responsive server method comprising:
controlling a wearable ring display to display a first color corresponding to a first mood or emotion at a first point in time; and
controlling the wearable ring display to display a second color corresponding to a second mood or emotion at a second point in time, wherein the first color is different than the second color, wherein the displayed ring color is associated with a current feeling.

7. The real time responsive server method according to claim 6, further comprising:
responsive to receiving a user ring input via a wearable ring button, controlling a network communication interface of a wearable ring device to transmit the user ring input to a responsive server;
receiving, via the network communication interface, a response to the transmitted user ring input from the server; and
responsive to receiving the response to the transmitted user ring input from the server, controlling the wearable ring display to display the first color corresponding to the first mood or the second color corresponding to the second mood based on the received response to the transmitted user ring input.

8. The real time responsive server method according to claim 7, wherein the controlling of the wearable ring display to display the first color corresponding to the first mood or the second color corresponding to the second mood based on the received response to the transmitted user ring input includes controlling the wearable ring display to change a color of the wearable ring display.

9. The real time responsive server method according to claim 7, wherein the controlling the wearable ring display to change a color of the wearable ring display includes outputting a notification indicating the color change.

10. The real time responsive server method according to claim 7, wherein the outputting of the notification indicating the color change includes at least one of: controlling the wearable ring display to flash, controlling the wearable ring display to pulse, or controlling the wearable ring to output haptic feedback.

11. The real time responsive server method according to claim 7, further comprising:
receiving, from the wearable ring device, a packet including raw digit activity (RDA);
identifying, based on the packet, a registered wearable device user (RWDU) associated with a wearable ring device that transmitted the packet;
storing, in the database, the RDA in association with the RWDU;
detecting, based on the stored RDA, one or more indications of stress; and determining, based on the one or more indications of stress, an emotional state of the RWDU.

12. The real time responsive server method according to claim 7, further comprising:
displaying, on a display coupled to an external electronic computing device, a graphical user interface (GUI);
monitoring, by the electronic computing device, user interactions with a button displayed on the GUI;
while monitoring the user interactions with the button, detecting a user interaction with the button; and
based on the detecting of the user interaction with the button, determining a mood of the user.

13. A non-transitory computer-readable medium comprising computer-executable instructions that, when executed by processing circuitry, cause the processing circuitry to perform the real time responsive server method according to claim 6.

14. A method performed by a wearable electronic ring device, the method comprising:
controlling a wearable ring display to display a first color corresponding to a first mood or emotion at a first point in time; and
controlling a wearable ring display to display a second color corresponding to a second mood or emotion at a second point in time, wherein the first color is different than the second color, wherein the displayed ring color is associated with a current feeling or mood.

15. The method according to claim 14, further comprising:
responsive to receiving a user ring input via a wearable ring button, controlling a network communication interface to transmit the user ring input to a responsive server;
receive, via the network communication interface, a response to the transmitted user ring input from the server; and
responsive to receiving the response to the transmitted user ring input from the server, control the wearable ring display to display the first color corresponding to the first mood or the second color corresponding to the second mood based on the received response to the transmitted user ring input.

16. The method according to claim 15, wherein the controlling of the wearable ring display to display the first color corresponding to the first mood or the second color corresponding to the second mood based on the received response to the transmitted user ring input includes controlling the wearable ring display to change a color of the wearable ring display.

17. The method according to claim 16, wherein the controlling the wearable ring display to change a color of the wearable ring display includes outputting a notification indicating the color change.

18. The method according to claim 17, wherein the outputting of the notification indicating the color change includes at least one of: controlling the wearable ring display to flash, controlling the wearable ring display to pulse, or controlling the wearable ring to output haptic feedback.

19. A non-transitory computer-readable medium comprising computer-executable instructions that, when executed by processing circuitry, cause the processing circuitry to perform the method according to claim 14.
